(19) United States
(12) Patent Application Publication
(10) Pub. No.: US 2010/0204224 A1
(43) Pub. Date: Aug. 12, 2010

(54) AZACYCLIC COMPOUNDS AS INHIBITORS OF SENSORY NEURONE SPECIFIC SODIUM CHANNELS

(75) Inventors: Richard Hamlyn, Cambridge (GB); Glyn Addison, Cambridge (GB); Christopher Geoffrey Earnshaw, Cambridge (GB); Harry Finch, Cambridge (GB); Mike Huckstep, Cambridge (GB); Rosemary Lynch, Cambridge (GB); Sarah Mellor, Cambridge (GB)

Correspondence Address: BANNER & WITCOFF, LTD., 1100 13th STREET, N.W., SUITE 1200, WASHINGTON, DC, 20005-4051, US

(73) Assignee: VERNALIS R & D LIMITED, Winnersh (GB)

(21) Appl. No.: 11/993,557

(22) PCT Filed: Jul. 7, 2006
(86) PCT No.: PCT/GB2006/002539

§ 371 Date: Apr. 1, 2010

(30) Foreign Application Priority Data
Jul. 7, 2005 (GB) 0514018.1

(51) Int. Cl. A61K 31/5377; C07D 471/08; A61K 31/4995; C07D 487/10; A61K 31/407; C07D 487/04; A61P 13/10; A61P 25/00

(52) U.S. Cl. 514/234.5; 544/349; 514/249; 544/119; 548/409; 514/409; 548/453; 514/412

(57) ABSTRACT

Compounds of the formula (I), and pharmaceutically acceptable salts thereof, are found to be antagonists of SNS sodium channels. They are therefore useful as analgesic and neuroprotective agents; wherein (1) represents (A), (B) or (C); R¹ represents: (a) -L-A or L′-A′ wherein L represents a bond or a C₁-C₆ alkyl, C₂-C₆ alkenyl or C₂-C₆ alkynyl moiety, A represents a phenyl, 5- to 10-membered heteroaryl, C₃-C₆ carbocyclyl or 5- to 10 membered heterocyclyl group, L′ represents a C₁-C₆ alkyl, C₂-C₆ alkenyl or C₂-C₆ alkynyl moiety, and A′ represents -Het-A or —X-A wherein Het represents —O—, —S— or NR′, and X represents —CO—, —SO—, SO₂—, —CO—O—, CO—S, CONR′, —O—CO—, —S—CO— or NR′—CO—, wherein R′ represents hydrogen or C₁-C₆ alkyl; (b) -L-A-A′ or -L-A-L-A wherein A′ is as defined above, each A is the same or different and is as defined above and each L is the same or different and is as defined above; (c) -A-Z-A wherein Z is -Het-L′, —X-L′, -L′-Het- or L′-X, wherein Het, L′ and X are as defined above and each A is the same or different and is as defined above; (d) -A-Het-Y or -A-X—Y wherein is [L′-Het]ₙ-L′, [L′-Het]ₙ-L′, -[L′-Het]ₙ-A, -L′-B-L′, -L′-B-A or -A-L-A wherein n is from 1 to 4 and B is —X—, —NR′—CO—NR′, —O—CO—NR′— or —NR′—CO—O, and wherein X and L are as defined above, each A is the same or different and is as defined above, each L′ is the same or different and is as defined above, each R′ is the same or different and is as defined above and each Het is the same or different and is as defined above; or (e) -L-CR(A)(A′) or L-CR(A)(L-A) wherein R is hydrogen or C₁-C₄ alkyl, A′ is as defined above, each L is the same or different and is as defined above and each A is the same or different and is as defined above; R² represents -L-A-, L′-A′, -L-A-A′ or L-A-L-A wherein L′ and A′ are as defined above, each L is the same or different and is as defined above and each A is the same or different and is as defined above J represents —NR³—, —O— or a direct bond wherein R³ represent s hydrogen C₁-C₆ alkyl, C₂-C₆ alkenyl or C₂-C₆ alkynyl; p is an integer from 1 to 3; q is 1 or 2; and one of E and E′ is —CH₂— and the other is a direct bond.

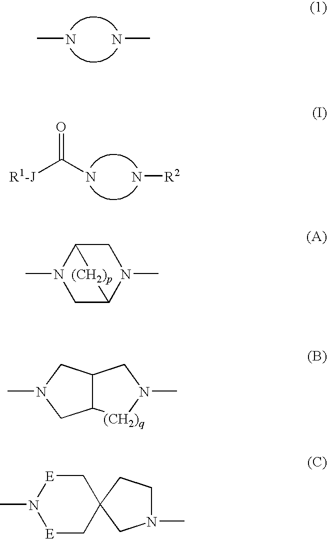

The present invention relates to inhibitors of the subtype of mammalian sodium channels known as Naᵥ1.8 or sensory neurone specific (SNS) channels. The Naᵥ1.8 channel is a 1,957 amino acid tetrodotoxin-insensitive voltage-gated sodium channel. The sodium channel, nucleic acid sequences coding for the channel, vectors, host cells and methods of identifying modulators, are taught in U.S. Pat. No. 6,451,554. The α-subunit gene corresponding to this ion channel is referred to as SCN10A. The channel is described in more detail in Akopian et al., (1996), 379, 257-262.

Mammalian ion channels are becoming increasingly well characterized, and progress in sodium channel research has been summarized recently in Anger et al, J. Med. Chem. (2001) 44, 115-137. Sodium channels are recognised as valid targets for pain therapeutics, and blockade of sodium channels can be useful in the treatment of a range of pain syndromes (see for example Black et al, Progress in Pain Research and Management (2001), 21 (Neuropathic Pain: Pathophysiology and Treatment), 19-36).

It has now surprisingly been found that compounds of the general formula (I) set out below act as inhibitors of sensory neurone specific sodium channels. Accordingly, the present invention provides the use, in the manufacture of a medicament for use in the treatment or prevention of a condition involving sodium ion flux through a sensory neurone specific channel of a sensory neurone, of a compound of the formula (I), or a pharmaceutically acceptable salt thereof

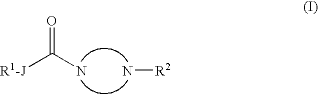

wherein:

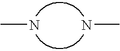

represents (A), (B) or (C)

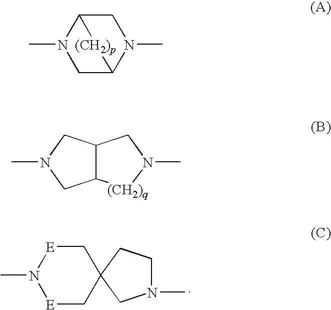

R¹ represents:

(a) -L-A or -L′-A′ wherein L represents a bond or a C₁-C₆ alkyl, C₂-C₆ alkenyl or C₂-C₆ alkynyl moiety, A represents a phenyl, 5- to 10-membered heteroaryl, C₃-C₆ carbocyclyl or 5- to 10-membered heterocyclyl group, L′ represents a C₁-C₆ alkyl, C₂-C₆ alkenyl or C₂-C₆ alkynyl moiety, and A′ represents -Het-A or —X-A wherein Het represents —O—, —S— or —NR′—, and X represents —CO—, —SO—, —SO₂—, —CO—O—, —CO—S—, —CONR′—, —O—CO—, —S—CO— or —NR′—CO—, wherein R′ represents hydrogen or C₁-C₆ alkyl;

(b) -L-A-A′ or -L-A-L-A wherein A′ is as defined above, each A is the same or different and is as defined above and each L is the same or different and is as defined above;

(c) -A-Z-A wherein Z is -Het-L′-, —X-L′-, -L′-Het- or -L′-X—, wherein Het, L′ and X are as defined above and each A is the same or different and is as defined above;

(d) -A-Het-Y or -A-X—Y wherein Y is -[L′-Het]ₙ-L′, -[L′-Het]ₙ-A, -L′-B-L′, -L′-B-A or -A-L-A wherein n is an integer from 1 to 4 and B is —X—, —NR′—CO—NR′—, —O—CO—NR′— or —NR′—CO—O—, and wherein X and L are as defined above, each A is the same or different and is as defined above, each L′ is the same or different and is as defined above, each R′ is the same or different and is as defined above and each Het is the same or different and is as defined above; or

(e) -L-CR(A)(A′) or -L-CR(A)(L-A) wherein R is hydrogen or C₁-C₄ alkyl, A′ is as defined above, each L is the same or different and is as defined above and each A is the same or different and is as defined above;

R² represents -L-A, -L′-A′, -L-A-A′ or -L-A-L-A wherein L′ and A′ are as defined above, each L is the same or different and is as defined above and each A is the same or different and is as defined above

J represents —NR³—, —O— or a direct bond wherein R³ represents hydrogen, C₁-C₆ alkyl, C₂-C₆ alkenyl or C₂-C₆ alkynyl;

p is an integer from 1 to 3;

q is 1 or 2; and

one of E and E′ is —CH₂— and the other is a direct bond;

wherein:

said phenyl, carbocyclyl, heterocyclyl and heteroaryl groups are optionally fused to a further cyclic moiety selected from phenyl, C₅-C₆ carbocyclyl, 5- to 6-membered heterocyclyl and 5- to 6-membered heteroaryl groups;

the phenyl, heteroaryl, carbocyclyl and heterocyclyl groups and moieties in the groups R¹ and R² are unsubstituted or substituted by one, two or three substituents which are the same or different and are selected from halogen, hydroxy, amino, thio, nitro, cyano, C₁-C₆ alkyl, C₂-C₆ alkenyl or -Het-L′, wherein Het and L′ are as defined above; and

the alkyl, alkenyl and alkynyl groups and moieties in R¹ to R³ are unsubstituted or substituted by one, two or three substituents which are the same or different and are selected from halogen, hydroxy, amino, thio and cyano substituents.

For the avoidance of doubt, when

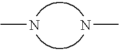

represents (A), (B) or (C)
the orientation of the groups (A), (B) and (C) is such that the left hand side of the depicted group is attached to the carbonyl moiety shown in formula (I). Thus, for example, when

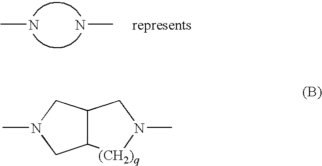

the compound of formula (I) is

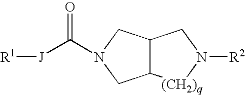

For the avoidance of doubt, when A′ represents —X-A, the orientation of the group X is such that the right hand side of the depicted moiety is attached to A. Thus, for example, when X is —CO—O—, the group —X-A is —CO—O-A.

For the avoidance of doubt, when R¹ represents -A-Z-A, the orientation of the group Z is such that the left hand side of the depicted moiety is attached to the divalent A group. Thus, for example, when Z is -Het-L′-, the group -A-Z-A is -A-Het-L′-A.

For the avoidance of doubt, when Z represents —X-L′-, the orientation of the group X is such that the right hand side of the depicted moiety is attached to L′. Thus, for example, when X is —CO—O—, the group —X-L′ is —CO—O-L.

For the avoidance of doubt, when Z represents -L′-X—, the orientation of the group X is such that the left hand side of the depicted moiety is attached to L′. Thus, for example, when X is —CO—O—, the group -L′-X— is -L′-CO—O—.

For the avoidance of doubt, when R¹ represents -A-X—Y, the orientation of the group X is such that the right hand side of the depicted moiety is attached to Y. Thus, for example, when X is —CO—O—, the group -A-X—Y is -A-CO—O—Y.

For the avoidance of doubt, when Y represents -L′-B-L′, the orientation of the group B is such that the right hand side of the depicted moiety is attached to the monovalent L′ group. Thus, for example, when B is —NR′—CO—O—, the group -L′-B-L′ is -L′-NR′—CO—O-L′.

For the avoidance of doubt, when Y represents -L′-B-A, the orientation of the group B is such that the right hand side of the depicted moiety is attached to A. Thus, for example, when B is —NR′—CO—O—, the group -L′-B-A is -L′-NR′—CO—O-A.

As used herein, a C₁-C₆ alkyl group or moiety is a linear or branched alkyl group or moiety containing from 1 to 6 carbon atoms, such as C₁-C₄ alkyl group or moiety, for example methyl, ethyl, n-propyl, i-propyl, n-butyl, i-butyl and t-butyl. Preferred C₁-C₆ alkyl groups are methyl, ethyl, n-propyl and n-butyl. A divalent alkyl moiety (or alkylene moiety) can be attached via the same carbon atom, by adjacent carbon atoms or by non-adjacent carbon atoms. Preferred divalent alkyl groups are methylene, 1,1-ethylene, 1,2-ethylene, 1,2-propylene and 1,3-propylene.

As used herein, a C₂-C₆ alkenyl group or moiety is a linear or branched alkenyl group or moiety containing from 2 to 6 carbon atoms, such as a C₂-C₄ alkenyl group or moiety, for example ethenyl, propenyl, butenyl, or —CH₂—CH═C(CH₃)₂. A preferred alkenyl group is propenyl. Typically, an alkenyl group or moiety is saturated except for one double bond. A divalent alkenyl moiety (or alkenylene moiety) can be attached via the same carbon atoms, via adjacent carbon atoms or via non-adjacent carbon atoms.

As used herein, a C₂-C₆ alkynyl group or moiety is a linear or branched alkynyl group or moiety containing from 2 to 6 carbon atoms, such as a C₂-C₄ alkynyl group or moiety, for example ethynyl, propynyl and butynyl. Typically, an alkynyl group or moiety is saturated except for one triple bond. A divalent alkynyl moiety (or alkynylene moiety) can be attached via the same carbon atom, via adjacent carbon atoms or via non-adjacent carbon atoms.

When a phenyl moiety is fused to a cyclic group, it is preferably fused to a further phenyl ring to form a napthyl group.

As used herein, a 5- to 10-membered heteroaryl group is a monocyclic 5- to 10-membered aromatic ring, such as a 5- or 6-membered ring, containing at least one heteroatom, for example 1, 2 or 3 heteroatoms, selected from O, S and N. Examples include pyridyl, pyrazinyl, pyrimidinyl, pyridazinyl, furanyl, thienyl, imidazolyl, pyrrolyl, triazolyl, oxadiazolyl, oxazolyl, isoxazyl, thiadiazolyl, isothiazolyl, thiazolyl and pyrazolyl groups. Pyridyl, thienyl, thiazolyl, pyrrolyl and imidazolyl groups are preferred.

When a 5- to 10-membered heteroaryl moiety is fused to a phenyl, 5- to 6-membered heteroaryl, C₅-C₆ carbocyclyl or 5- to 6-membered heterocyclyl group, it is preferably a 5- to 6-membered heteroaryl moiety fused to a phenyl, 5- to 6-membered heteroaryl, C₅-C₆ carbocyclyl or 5- to 6-membered heterocyclyl group. When a 5- to 10-membered heteroaryl moiety is fused to a cyclic group, it is preferably fused to a phenyl group. Examples of such fused groups include a pyrrolyl moiety that is fused to a phenyl group to form an indolyl group.

As used herein, a halogen is typically fluorine, chlorine, bromine or iodine and is preferably fluorine, chlorine or bromine.

As used herein, a C₁-C₂ haloalkyl group is typically a said C₁-C₂ alkyl group substituted by one or more said halogen atoms. Typically, it is substituted by 1, 2 or 3 said halogen atoms. Preferred haloalkyl groups include perhaloalkyl groups such as —CX₃ wherein X is a said halogen atom. A particularly preferred haloalkyl group is —CF₃.

As used herein, a C₃-C₆ carbocyclyl group or moiety is a monocyclic, non-aromatic saturated or unsaturated hydrocarbon ring, having from 3 to 6 carbon atoms. Preferably it is a saturated group, i.e. a C₃-C₆ cycloalkyl group. Examples include cyclopropyl, cyclobutyl, cyclopentyl and cyclohexyl. Preferred carbocyclyl groups are cyclopentyl and cyclohexyl.

When a C₃-C₆ carbocyclyl moiety is fused to a phenyl, 5- to 6-membered heteroaryl, C₅-C₆ carbocyclyl or 5- to 6-membered heterocyclyl group, it is preferably a C₅-C₆ carbocyclyl moiety fused to a phenyl, 5- to 6-membered heteroaryl, C₅-C₆ carbocyclyl or 5- to 6-membered heterocyclyl group. When a C₃-C₆ carbocyclyl moiety is fused to a cyclic group, it is preferably fused to a phenyl group. Examples of such fused groups include a cyclopentyl moiety that is fused to a phenyl group to form a dihydroindenyl group and a cyclohexyl group that is fused to a phenyl group to form a tetrahydronaphthalenyl group.

As used herein, a 5- to 10-membered heterocyclyl group or moiety is a monocyclic, non-aromatic, saturated or unsaturated C₅-C₁₀ carbocyclic ring in which one or more, for example 1, 2 or 3, of the carbon atoms are replaced by a moiety selected from N, O, S, C(O), S(O) and S(O)₂. Preferably, only one or two carbon atoms are replaced with a —C(O)—, —S(O)— or —S(O)₂— moiety. More preferably, a 5- to 10-membered heterocyclyl group or moiety is a monocyclic, non-aromatic, saturated or unsaturated C₅-C₁₀ carbocyclic ring in which one or more, for example 1, 2 or 3, of the carbon atoms are replaced by a heteroatom selected from N, O and S.

Saturated heterocyclyl groups are preferred. Examples of suitable heterocyclyl groups include piperidinyl, piperazinyl, tetrahydropyranyl, dioxanyl, tetrahydrothiopyranyl, dithianyl, morpholinyl, thiomorpholinyl, S-oxo-thiomorpholino, S,S-dioxo-thiomorpholino, pyrrolidinyl, imidazolidinyl, pyrazolidinyl, tetrahydrofuranyl, dioxolanyl, tetrahydrothiophenyl, dithiolanyl, thiazolidinyl, oxazolidinyl, pyrrolidinonyl and pyrrolidin-2,5-dionyl groups. Preferred heterocyclyl groups are tetrahydrofuranyl, pyrrolidinyl, tetrahydropyranyl, morpholinyl, pyrrolidinonyl and piperidinyl groups.

When a 5- to 10-membered heterocyclyl moiety is fused to a phenyl, 5- to 6-membered heteroaryl, C₅-C₆ carbocyclyl or 5- to 6-membered heterocyclyl group, it is preferably a 5- to 6-membered heterocyclyl moiety fused to a phenyl, 5- to 6-membered heteroaryl, C₅-C₆ carbocyclyl or 5- to 6-membered heterocyclyl group. When a 5- to 10-membered heterocyclyl moiety is fused to a cyclic group, it is preferably fused to a phenyl group.

Typically, R′ represents hydrogen or C₁-C₂ alkyl. Preferably, R′ represents hydrogen or methyl.

Typically, Het represents —O— or —NR′— wherein R′ is as defined above. Preferably, Het represents —O— or —NH— or —NMe-.

Typically, X represents —CO—, —CO—O—, —CO—S— or —CONR′—, wherein R′ is as defined above. Preferably, X represents —CO— or —CO—O—. More preferably, X represents —CO—.

Typically, L represents a bond or a C₁-C₆ alkyl or C₂-C₆ alkenyl moiety. Preferably, L represents a bond or a C₁-C₄ alkyl moiety.

Typically, L′ represents a C₁-C₆ alkyl or C₂-C₆ alkenyl moiety. Preferably, L′ represents a C₁-C₄ alkyl or C₂-C₄ alkenyl moiety.

Typically, the phenyl, heteroaryl, heterocyclyl and carbocyclyl groups and moieties in the groups R¹ and R² are unsubstituted or substituted by one, two or three substituents which are the same or different and are selected from fluorine, chlorine, bromine, cyano, C₁-C₆ alkyl, C₂-C₆ alkenyl or -Het-L′, wherein Het and L′ are as defined above, the alkyl, alkenyl and alkynyl substituents being unsubstituted or substituted by one, two or three further substituents which are the same or different and are selected from fluorine, chlorine, bromine, hydroxy, amino and thio substituents.

Preferably, the phenyl, heteroaryl, heterocyclyl and carbocyclyl groups and moieties in the groups R¹ and R² are unsubstituted or are substituted by one or two unsubstituted substituents which are the same or different and are selected from fluorine, chlorine, bromine, cyano, C₁-C₄ alkyl, C₂-C₄ alkenyl, C₁-C₂ haloalkyl, —O—(C₁-C₄ alkyl) or —O—(C₂-C₄ alkenyl). Typically, when a phenyl, heteroaryl, heterocyclyl and carbocyclyl group or moiety is substituted by either cyano or nitro, each cyclic group or moiety only carries a single cyano or nitro group.

Typically, the alkyl, alkenyl and alkynyl groups and moieties in R¹ to R³ are unsubstituted or substituted by a single hydroxy or cyano substituent or by one, two or three substituents selected from fluorine or chlorine. Preferably, the alkyl, alkenyl and alkynyl groups and moieties in R¹ to R³ are unsubstituted or substituted by a single hydroxy or cyano substituent or by one, two or three fluorine substituents. In a preferred embodiment, the alkyl, alkenyl and alkynyl groups and moieties in R¹ to R⁴ are unsubstituted.

Typically, A represents a phenyl, 5- to 6-membered heteroaryl, C₃-C₆ carbocyclyl or 5- to 6-membered heterocyclyl group, said group being optionally fused to a phenyl, 5- to 6-membered heteroaryl, C₅-C₆ carbocyclyl or 5- to 6-membered heterocyclyl moiety. Preferably, A represents a phenyl, 5- to 6-membered heteroaryl, C₃-C₆ carbocyclyl or 5- to 6-membered heterocyclyl group, said group being optionally fused to a phenyl moiety. More preferably, A represents a phenyl, pyridyl, thienyl, thiazolyl, imidazolyl, cyclopentyl, dihydroindenyl, tetrahydronaphthalenyl, tetrahydrofuranyl, pyrrolidinyl, tetrahydropyranyl, morpholinyl, pyrrolidinonyl, piperidinyl or indolyl group.

Typically, when R¹ comprises a group A, A is a phenyl, 5- to 6-membered heteroaryl, C₃-C₆ carbocyclyl or 5- to 6-membered heterocyclyl group which is optionally fused to a phenyl ring. Preferably, when R¹ comprises a group A, A is a phenyl, pyridyl, thiazolyl, imidazolyl, cyclopentyl, tetrahydrofuranyl, pyrrolidinyl, tetrahydropyranyl, morpholinyl, pyrrolidinonyl, piperidinyl or indolyl group. Typically, when R¹ comprises a group A which is a cyclic moiety fused to a further cyclic moiety, R¹ comprises only one such fused group.

Typically, when R² comprises a group A, A is a phenyl, 5- to 6-membered heteroaryl, C₃-C₆ carbocyclyl or 5- to 6-membered heterocyclyl group which is optionally fused to a phenyl ring. Preferably, when R² comprises a group A, A is a phenyl, thienyl, tetrahydronaphthalenyl or dihydroindenyl group. Typically, when R² comprises a group A which is a cyclic moiety fused to a further cyclic moiety, R² comprises only one such fused group.

Typically, A′ represents -Het-A or —X-A, wherein Het is —O— and X is —C(O)— or —C(O)—O— and A is as defined above. Preferably, A′ represents —O-A or —C(O)-A wherein A is a phenyl, 5- to 6-membered heteroaryl, C₃-C₆ carbocyclyl or 5- to 6-membered heterocyclyl group.

Typically, Z is -Het-L′- or —X-L′-, wherein Het, X and L′ are as defined above. Preferably, Z is —O—(C₁-C₂ alkyl)-.

Typically, n is an integer from 1 to 3. Preferably, n is 1 or 2.

Typically, B is —NR′—CO—NR′—, —O—CO—NR′— or —NR′—CO—O— wherein R′ is as defined above. Preferably, B is —NR′—CO—O—, wherein R′ is as defined above. More preferably, B is —NH—CO—O—.

Typically, Y is -[L′-Het]ₙ-L′, -L′-B-L′ or -A-L-A wherein n, L and B are as defined above, each L′ is the same or different and is as defined above, each Het is the same or different and is as defined above and each A is the same or different and is as defined above. Preferably, Y is -[L′-Het]ₙ-L′, -L′-B-L′ or -A-L-A, wherein n is 1 or 2, each L′ is the same or different and is a C₁-C₄ alkyl or C₂-C₄ alkenyl moiety, each Het is the same or different and is —O—, —NH— or —NMe-, B is —NH—CO—O—, each A is the same or different and is a phenyl or piperidinyl group and L is a C₁-C₂ alkyl moiety.

Typically, R is hydrogen or C₁-C₂ alkyl. Preferably, R is hydrogen.

Typically, when R¹ represents -L-A, L is a bond or a C₁-C₄ alkyl moiety and A is a phenyl, 5- to 6-membered heteroaryl, C₃-C₆ carbocyclyl or 5- to 6-membered heterocyclyl group which is optionally fused to a phenyl ring. Preferably, when R¹ represents -L-A, L is a bond or a C₁-C₂ alkyl moiety and A is a phenyl or indolyl group.

Typically, when R¹ represents -L-A-A′, A′ is -Het-A or —X-A wherein Het and X are as defined above, L is a bond or C₁-C₄ alkyl moiety and each A is the same or different and is a phenyl, 5- to 6-membered heteroaryl, C₃-C₆ carbocyclyl or 5- to 6-membered heterocyclyl group. Preferably, when R¹ represents -L-A-A′, A′ is —O-A or —C(O)-A, L is a bond or a C₁-C₂ alkyl moiety and each A is the same or different and is a phenyl, pyridyl, tetrahydropyranyl or cyclopentyl group. In a preferred embodiment, when R¹ represents -L-A-A′ the divalent A group is a phenyl or pyridyl moiety.

Typically, when R¹ represents -L-A-L-A, each A is the same or different and is a phenyl, 5- to 6-membered heteroaryl, C₃-C₆ carbocyclyl or 5- to 6-membered heterocyclyl group and each L is the same or different and is a bond or a C₁-C₄ alkyl moiety. Preferably, when R¹ represents -L-A-L-A, each A is a phenyl group and each L is the same or different and is a bond or a C₁-C₂ alkyl moiety.

Typically, when R¹ represents -A-Z-A, each A is the same or different and is a phenyl, 5- to 6-membered heteroaryl, C₃-C₆ carbocyclyl or 5- to 6-membered heterocyclyl group and Z is -Het-L′- or —X-L′-, wherein L′ is a C₁-C₄ alkyl moiety and Het and X are as defined above. Preferably, when R¹ represents -A-Z-A, each A is the same or different and is a phenyl, pyridyl, thiazolyl, imidazolyl, tetrahydrofuranyl, tetrahydropyranyl, pyrrolidinyl, morpholinyl or pyrrolidinonyl group and Z is —O—(C₁-C₂ alkyl)-. In a preferred embodiment, when R¹ represents -A-Z-A, the divalent A group is a phenyl moiety.

Typically, when R¹ represents -A-Het-Y, Y is -[L′-Het]ₙ-L′, -L′-B-L′ or -A-L-A, each A is the same or different and is a phenyl, 5- to 6-membered heteroaryl, C₃-C₆ carbocyclyl or 5- to 6-membered heterocyclyl group, each L′ is the same or different and is a C₁-C₆ alkyl or C₂-C₆ alkenyl moiety, n is as defined above, L is a bond or C₁-C₄ alkyl moiety, B is as defined above and each Het is the same or different and is as defined above. Preferably, when R¹ represents -A-Het-Y, A is a phenyl group, Het is —O— and Y is -[L′-Het]ₙ-L′, -L′-B-L′ or -A-L-A, wherein n is 1 or 2, each L′ is the same or different and is a C₁-C₄ alkyl or C₂-C₄ alkenyl moiety, each Het is the same or different and is —O—, —NH— or —NMe-, B is —NH—CO—O—, each A is the same or different and is a phenyl or piperidinyl group and L is a C₁-C₂ alkyl moiety.

Typically, R¹ represents:

(a) -L-A wherein L and A are as defined above;

(b) -L-A-A′ or -L-A-L-A wherein A′ is as defined above, each L is the same or different and is as defined above and each A is the same or different and is as defined above;

(c) -A-Z-A wherein Z is as defined above and each A is the same or different and is as defined above; or

(d) -A-Het-Y wherein A, Het and Y are as defined above.

Preferably, R¹ represents:

(a) -L-A wherein L is a bond or a C₁-C₂ alkyl moiety and A is a phenyl or indolyl group;

(b) -L-A-A′ wherein A′ is —O-A or —C(O)-A and L is a bond or a C₁-C₂ alkyl moiety, each A is the same or different and is a phenyl, pyridyl, tetrahydropyranyl or cyclopentyl group, or -L-A-L-A wherein A is a phenyl group and each L is the same or different and is a bond or a C₁-C₂ alkyl moiety;

(c) -A-Z-A wherein each A is the same or different and is a phenyl, pyridyl, thiazolyl, imidazolyl, tetrahydrofuranyl, tetrahydropyranyl, pyrrolidinyl, morpholinyl or pyrrolidinonyl group and Z is —O—(C₁-C₂ alkyl)-; or

(d) -A-Het-Y wherein A is a phenyl group, Het is —O— and Y is -[L′-Het]ₙ-L′, -L′-B-L′ or -A-L-A, wherein n is 1 or 2, each L′ is the same or different and is a C₁-C₄ alkyl or C₂-C₄ alkenyl moiety, each Het is the same or different and is —O—, —NH— or —NMe-, B is —NH—CO—O—, each A is the same or different and is a phenyl or piperidinyl group and L is a C₁-C₂ alkyl moiety.

Typically, when R² represents -L-A, L is a bond or a C₁-C₄ alkyl moiety and A is a phenyl, 5- to 6-membered heteroaryl, C₃-C₆ carbocyclyl or 5- to 6-membered heterocyclyl group which is optionally fused to a phenyl group. Preferably, when R² represents -L-A, L is a bond or a C₁-C₄ alkyl moiety and A is a phenyl, thienyl, pyridyl, dihydroindenyl or tetrahydronaphthalenyl group.

Typically, R² represents -L-A or -L-A-A′ wherein L, A and A′ are as defined above. Preferably, R² represents -L-A, wherein L is a bond or C₁-C₄ alkyl moiety and A is a phenyl, 5- to 6-membered heteroaryl, C₃-C₆ carbocyclyl or 5- to 6-membered heterocyclyl group which is optionally fused to a phenyl group. More preferably, R² represents -L-A wherein L is a bond or a C₁-C₄ alkyl moiety and A is a phenyl, thienyl, pyridyl, dihydroindenyl or tetrahydronaphthalenyl group.

Typically, R³ represents hydrogen or C₁-C₄ alkyl. Preferably, R² represents hydrogen or methyl.

Typically, J is a direct bond or —NR³—, wherein R³ is as defined above. Preferably, J is a direct bond, —NH— or —NMe-.

Typically, p is 1.

Typically, q is 1.

Typically, E represents —CH₂— and E′ represents a direct bond;

Typically,

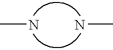

represents (A*), (B*) or (C*)

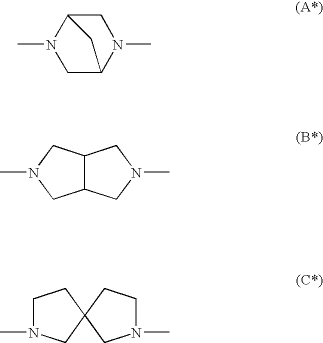

Preferred compounds of formula (I) are those compounds wherein:

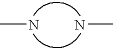

represents (A*), (B*) or (C*)

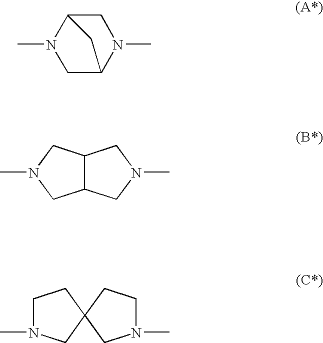

R¹ represents:

(a) -L-A wherein L is a bond or a C₁-C₄ alkyl moiety and A is a phenyl, 5- to 6-membered heteroaryl, C₃-C₆ carbocyclyl or 5- to 6-membered heterocyclyl group which is optionally fused to a phenyl ring;

(b) -L-A-A′ or -L-A-L-A wherein A′ is -Het-A or —X-A wherein Het is —O— or —NR′—, X is —CO—, —CO—O—, —CO—S— or —CONR′—, R′ is hydrogen or C₁-C₂ alkyl, each L is the same or different and is as defined above as defined above and each A is the same or different and is a phenyl, 5- to 6-membered heteroaryl, C₃-C₆ carbocyclyl or 5- to 6-membered heterocyclyl group;

(c) -A-Z-A wherein each A is the same or different and is a phenyl, 5- to 6-membered heteroaryl, C₃-C₆ carbocyclyl or 5- to 6-membered heterocyclyl group and Z is -Het-L′- or —X-L′-, wherein L′ is a C₁-C₄ alkyl moiety and Het and X are as defined above; and

(d) -A-Het-Y wherein Y is -[L′-Het]ₙ-L′, -L′-B-L′ or -A-L-A, each A is the same or different and is a phenyl, 5- to 6-membered heteroaryl, C₃-C₆ carbocyclyl or 5- to 6-membered heterocyclyl group, each L′ is the same or different and is a C₁-C₆ alkyl or C₂-C₆ alkenyl moiety, n is an integer from 1 to 3, L is as defined above, B is —NR′—CO—NR′—, —O—CO—NR′— or —NR′—CO—O—, R′ is as defined above and each Het is the same or different and is as defined above;

R² represents -L-A or L-A-A′ wherein L and A′ are as defined above and each A is the same or different and is a phenyl, 5- to 6-membered heteroaryl, C₃-C₆ carbocyclyl or 5- to 6-membered heterocyclyl group which is optionally fused to a phenyl ring; and

J is a direct bond or —NR³— wherein R³ represents hydrogen or C₁-C₄ alkyl; wherein:

said phenyl, heteroaryl, heterocyclyl and carbocyclyl groups and moieties in the groups R¹ and R² are unsubstituted or substituted by one, two or three substituents which are the same or different and are selected from fluorine, chlorine, bromine, cyano, C₁-C₆ alkyl, C₂-C₆ alkenyl or -Het-L′, wherein Het and L′ are as defined above, the alkyl, alkenyl and alkynyl substituents being unsubstituted or substituted by one, two or three further substituents which are the same or different and are selected from fluorine, chlorine, bromine, hydroxy, amino and thio substituents.

the alkyl, alkenyl and alkynyl groups and moieties in R¹ to R³ are unsubstituted or substituted by a single hydroxy or cyano substituent or by one, two or three substituents selected from fluorine or chlorine.

More preferred compounds of the present invention are those wherein:

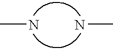

represents (A*), (B*) or (C*)

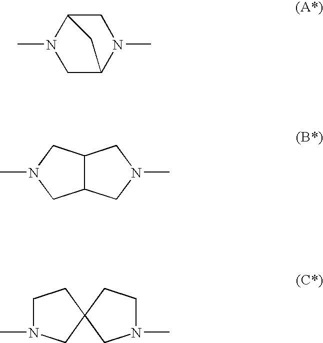

R¹ represents:

(a) -L-A wherein L is a bond or a C₁-C₂ alkyl moiety and A is a phenyl or indolyl group;

(b) -L-A-A′ wherein A′ is —O-A or —C(O)-A, L is a bond or a C₁-C₂ alkyl moiety and each A is the same or different and is a phenyl, pyridyl, tetrahydropyranyl or cyclopentyl group or -L-A-L-A wherein each A is a phenyl group and each L is the same or different and is a bond or a C₁-C₂ alkyl moiety;

(c) -A-Z-A wherein each A is the same or different and is a phenyl, pyridyl, thiazolyl, imidazolyl, tetrahydrofuranyl, tetrahydropyranyl, pyrrolidinyl, morpholinyl or pyrrolidinonyl group and Z is —O—(C₁-C₂ alkyl)-; and

(d) -A-Het-Y wherein A is a phenyl ring, Het is —O— and Y is -[L′-Het]ₙ-L′, -L′-B-L′ or -A-L-A, wherein n is 1 or 2, each L′ is the same or different and is a C₁-C₄ alkyl or C₂-C₄ alkenyl moiety, each Het is the same or different and is —O—, —NH— or —NMe-, B is —NH—CO—O—, each A is the same or different and is a phenyl or piperidinyl group and L is a C₁-C₂ alkyl moiety;

R² represents -L-A wherein L is a bond or a C₁-C₄ alkyl moiety and A is a phenyl, thienyl, pyridyl, dihydroindenyl or tetrahydronaphthalenyl group;

J is a direct bond, —NH— or —NMe-,

wherein:

the phenyl, heteroaryl, heterocyclyl and carbocyclyl groups and moieties in the groups R¹ and R² are unsubstituted or are substituted by one or two unsubstituted substituents which are the same or different and are selected from fluorine, chlorine, bromine, cyano, C₁-C₄ alkyl, C₂-C₄ alkenyl, C₁-C₂ haloalkyl, —O—(C₁-C₄ alkyl) or —O—(C₂-C₄ alkenyl); and

the alkyl, alkenyl and alkynyl groups and moieties in R¹ to R³ are unsubstituted or substituted by a single hydroxy or cyano substituent or by one, two or three fluorine substituents.

Examples of preferred compounds of the invention include:
5-(3-Methyl-thiophen-2-ylmethyl)-2,5-diaza-bicyclo[2.2.1]heptane-2-carboxylic acid(4-phenoxy-phenyl)-amide;
5-(3-Methyl-thiophen-2-ylmethyl)-2,5-diaza-bicyclo[2.2.1]heptane-2-carboxylic acid(6-phenoxy-pyridin-3-yl)-amide;
5-(3-Methyl-thiophen-2-ylmethyl)-2,5-diaza-bicyclo[2.2.1]heptane-2-carboxylic acid(4-butoxy-phenyl)-amide;
5-(1-o-Tolyl-ethyl)-2,5-diaza-bicyclo[2.2.1]heptane-2-carboxylic acid(4-phenoxy-phenyl)-amide;
5-(1-o-Tolyl-ethyl)-2,5-diaza-bicyclo[2.2.1]heptane-2-carboxylic acid(4-butoxy-phenyl)-amide;
5-(1-o-Tolyl-ethyl)-2,5-diaza-bicyclo[2.2.1]heptane-2-carboxylic acid(4-trifluoromethyl-phenyl)-amide;
5-[1-(2-Chloro-4-fluoro-phenyl)-ethyl]-2,5-diaza-bicyclo[2.2.1]heptane-2-carboxylic acid(4-phenoxy-phenyl)-amide;
5-(1-o-Tolyl-ethyl)-2,5-diaza-bicyclo[2.2.1]heptane-2-carboxylic acid(3-phenoxy-phenyl)-amide;
5-[1-(2,6-Difluoro-phenyl)-ethyl]-2,5-diaza-bicyclo[2.2.1]heptane-2-carboxylic acid(4-phenoxy-phenyl)-amide;
5-[1-(2,6-Difluoro-phenyl)-ethyl]-2,5-diaza-bicyclo[2.2.1]heptane-2-carboxylic acid(4-phenoxy-phenyl)-amide;
5-[1-(2-Chloro-phenyl)-ethyl]-2,5-diaza-bicyclo[2.2.1]heptane-2-carboxylic acid(4-phenoxy-phenyl)-amide;
5-[1-(2-Chloro-phenyl)-ethyl]-2,5-diaza-bicyclo[2.2.1]heptane-2-carboxylic acid(4-phenoxy-phenyl)-amide;
5-[1-(2,6-Difluoro-phenyl)-ethyl]-2,5-diaza-bicyclo[2.2.1]heptane-2-carboxylic acid(4-trifluoromethyl-phenyl)-amide;
5-[1-(3-Methyl-thiophen-2-yl)-ethyl]-2,5-diaza-bicyclo[2.2.1]heptane-2-carboxylic acid(4-phenoxy-phenyl)-amide;
5-[1-(3-Methyl-thiophen-2-yl)-ethyl]-2,5-diaza-bicyclo[2.2.1]heptane-2-carboxylic acid(4-trifluoromethyl-phenyl)-amide;
5-(1-o-Tolyl-ethyl)-2,5-diaza-bicyclo[2.2.1]heptane-2-carboxylic acid {4-[2-(2-ethoxy-ethoxy)-ethoxy]-phenyl}-amide;
5-(1-o-Tolyl-ethyl)-2,5-diaza-bicyclo[2.2.1]heptane-2-carboxylic acid[4-(2-ethoxy-ethoxy)-phenyl]-amide;
5-(1-o-Tolyl-ethyl)-2,5-diaza-bicyclo[2.2.1]heptane-2-carboxylic acid {4-[2-(2-methoxy-ethoxy)-ethoxy]-phenyl}-amide;
5-(1-o-Tolyl-ethyl)-2,5-diaza-bicyclo[2.2.1]heptane-2-carboxylic acid[4-(2-methoxy-ethoxy)-phenyl]-amide;
5-(1-o-Tolyl-ethyl)-2,5-diaza-bicyclo[2.2.1]heptane-2-carboxylic acid(4-allyloxy-phenyl)-amide;
5-(1-o-Tolyl-ethyl)-2,5-diaza-bicyclo[2.2.1]heptane-2-carboxylic acid[4-(2-dimethylamino-ethoxy)-phenyl]-amide;
5-(1-o-Tolyl-ethyl)-2,5-diaza-bicyclo[2.2.1]heptane-2-carboxylic acid[4-(tetrahydro-pyran-4-yloxy)-phenyl]-amide;
5-(1-o-Tolyl-ethyl)-2,5-diaza-bicyclo[2.2.1]heptane-2-carboxylic acid {4-[2-(2-oxo-pyrrolidin-1-yl)-ethoxy]-phenyl}-amide;
5-(1-o-Tolyl-ethyl)-2,5-diaza-bicyclo[2.2.1]heptane-2-carboxylic acid(4-cyclopentyloxy-phenyl)-amide;
5-(1-o-Tolyl-ethyl)-2,5-diaza-bicyclo[2.2.1]heptane-2-carboxylic acid[4-(tetrahydro-pyran-2-ylmethoxy)-phenyl]-amide;
5-(1-o-Tolyl-ethyl)-2,5-diaza-bicyclo[2.2.1]heptane-2-carboxylic acid[4-(tetrahydrofuran-2-ylmethoxy)-phenyl]-amide;
5-(1-o-Tolyl-ethyl)-2,5-diaza-bicyclo[2.2.1]heptane-2-carboxylic acid[4-(3-methoxy-propoxy)-phenyl]-amide;
[2-(4-{[5-(1-o-Tolyl-ethyl)-2,5-diaza-bicyclo[2.2.1]heptane-2-carbonyl]-amino}-phenoxy)-ethyl]-carbamic acid allyl ester;
5-(1-o-Tolyl-ethyl)-2,5-diaza-bicyclo[2.2.1]heptane-2-carboxylic acid[4-(4-fluoro-phenoxy)-phenyl]-amide;
5-(1-o-Tolyl-ethyl)-2,5-diaza-bicyclo[2.2.1]heptane-2-carboxylic acid(4-phenoxy-phenyl)-amide;
5-(1-o-Tolyl-ethyl)-2,5-diaza-bicyclo[2.2.1]heptane-2-carboxylic acid(4-benzyloxy-phenyl)-amide;
5-(1-o-Tolyl-ethyl)-2,5-diaza-bicyclo[2.2.1]heptane-2-carboxylic acid(4-phenoxy-phenyl)-amide;
5-(1-o-Tolyl-ethyl)-2,5-diaza-bicyclo[2.2.1]heptane-2-carboxylic acid(4-benzyloxy-phenyl)-amide;
5-(1-o-Tolyl-ethyl)-2,5-diaza-bicyclo[2.2.1]heptane-2-carboxylic acid(4-benzyl-phenyl)-amide;
5-(1-o-Tolyl-ethyl)-2,5-diaza-bicyclo[2.2.1]heptane-2-carboxylic acid(4-benzyl-phenyl)-amide;
2-(4-Phenoxy-phenyl)-1-[5-(1-o-tolyl-ethyl)-2,5-diaza-bicyclo[2.2.1]hept-2-yl]-ethanone;
2-(4-Phenoxy-phenyl)-1-[5-(1-o-tolyl-ethyl)-2,5-diaza-bicyclo[2.2.1]hept-2-yl]-ethanone;
5-(1-o-Tolyl-ethyl)-2,5-diaza-bicyclo[2.2.1]heptane-2-carboxylic acid biphenyl-4-ylamide;
5-(1-o-Tolyl-ethyl)-2,5-diaza-bicyclo[2.2.1]heptane-2-carboxylic acid biphenyl-4-ylamide;
5-(1-o-Tolyl-ethyl)-2,5-diaza-bicyclo[2.2.1]heptane-2-carboxylic acid[4-(1-benzyl-piperidin-4-yloxy)-phenyl]-amide;
5-(1-o-Tolyl-ethyl)-2,5-diaza-bicyclo[2.2.1]heptane-2-carboxylic acid[4-(2-pyrrolidin-1-yl-ethoxy)-phenyl]-amide;
5-(1-o-Tolyl-ethyl)-2,5-diaza-bicyclo[2.2.1]heptane-2-carboxylic acid {4-[2-(4-methyl-thiazol-5-yl)-ethoxy]-phenyl}-amide;
5-(1-o-Tolyl-ethyl)-2,5-diaza-bicyclo[2.2.1]heptane-2-carboxylic acid[4-(2-imidazol-1-yl-ethoxy)-phenyl]amide;
5-(1-o-Tolyl-ethyl)-2,5-diaza-bicyclo[2.2.1]heptane-2-carboxylic acid[4-(2-morpholin-4-yl-ethoxy)-phenyl]-amide;
5-(1-o-Tolyl-ethyl)-2,5-diaza-bicyclo[2.2.1]heptane-2-carboxylic acid[4-(2-pyridin-2-yl-ethoxy)-phenyl]-amide;
5-[1-(4-Fluoro-phenyl)-ethyl]-2,5-diaza-bicyclo[2.2.1]heptane-2-carboxylic acid(4-trifluoromethyl-phenyl)-amide;
5-[1-(4-Fluoro-phenyl)-ethyl]-2,5-diaza-bicyclo[2.2.1]heptane-2-carboxylic acid(3-trifluoromethyl-phenyl)-amide;
5-[1-(4-Fluoro-phenyl)-ethyl]-2,5-diaza-bicyclo[2.2.1]heptane-2-carboxylic acid(4-phenoxy-phenyl)-amide;
5-[1-(4-Fluoro-phenyl)-ethyl]-2,5-diaza-bicyclo[2.2.1]heptane-2-carboxylic acid(3-phenoxy-phenyl)-amide;
5-[1-(4-Fluoro-phenyl)-ethyl]-2,5-diaza-bicyclo[2.2.1]heptane-2-carboxylic acid(3-trifluoromethyl-phenyl)-amide;
5-[1-(4-Fluoro-phenyl)-ethyl]-2,5-diaza-bicyclo[2.2.1]heptane-2-carboxylic acid(4-phenoxy-phenyl)-amide;
5-[1-(4-Fluoro-phenyl)-ethyl]-2,5-diaza-bicyclo[2.2.1]heptane-2-carboxylic acid(3-phenoxy-phenyl)-amide;
5-[1-(4-Fluoro-phenyl)-ethyl]-2,5-diaza-bicyclo[2.2.1]heptane-2-carboxylic acid(4-trifluoromethyl-phenyl)-amide;
5-[1-(2-Trifluoromethyl-phenyl)-ethyl]-2,5-diaza-bicyclo[2.2.1]heptane-2-carboxylic acid(4-trifluoromethyl-phenyl)-amide;
5-[1-(2-Trifluoromethyl-phenyl)-ethyl]-2,5-diaza-bicyclo[2.2.1]heptane-2-carboxylic acid(3-trifluoromethyl-phenyl)-amide;
5-[1-(2-Trifluoromethyl-phenyl)-ethyl]-2,5-diaza-bicyclo[2.2.1]heptane-2-carboxylic acid(4-trifluoromethyl-phenyl)-amide;
5-[1-(2-Trifluoromethyl-phenyl)-ethyl]-2,5-diaza-bicyclo[2.2.1]heptane-2-carboxylic acid(3-trifluoromethyl-phenyl)-amide;
5-(1-Methyl-2-o-tolyl-ethyl)-2,5-diaza-bicyclo[2.2.1]heptane-2-carboxylic acid(4-phenoxy-phenyl)-amide;
5-(1-Methyl-2-o-tolyl-ethyl)-2,5-diaza-bicyclo[2.2.1]heptane-2-carboxylic acid(4-trifluoromethyl-phenyl)-amide;
5-[1-(2-Trifluoromethyl-phenyl)-ethyl]-2,5-diaza-bicyclo[2.2.1]heptane-2-carboxylic acid(4-phenoxy-phenyl)-amide;
5-[1-(2-Trifluoromethyl-phenyl)-ethyl]-2,5-diaza-bicyclo[2.2.1]heptane-2-carboxylic acid(3-phenoxy-phenyl)-amide;
5-[1-(2-Tri fluoromethyl-phenyl)-ethyl]-2,5-diaza-bicyclo[2.2.1]heptane-2-carboxylic acid(4-phenoxy-phenyl)-amide;
5-[1-(2-Trifluoromethyl-phenyl)-ethyl]-2,5-diaza-bicyclo[2.2.1]heptane-2-carboxylic acid(3-phenoxy-phenyl)-amide;
5-[1-(4-Fluoro-2-methyl-phenyl)-ethyl]-2,5-diaza-bicyclo[2.2.1]heptane-2-carboxylic acid(4-phenoxy-phenyl)-amide;
5-[1-(4-Fluoro-2-methyl-phenyl)-ethyl]-2,5-diaza-bicyclo[2.2.1]heptane-2-carboxylic acid(4-trifluoromethyl-phenyl)-amide;
5-(1-o-Tolyl-ethyl)-2,5-diaza-bicyclo[2.2.1]heptane-2-carboxylic acid(4-benzoyl-phenyl)-amide;
5-(1-Methyl-2-o-tolyl-ethyl)-2,5-diaza-bicyclo[2.2.1]heptane-2-carboxylic acid(3-trifluoromethyl-phenyl)-amide;
5-(1-o-Tolyl-ethyl)-2,5-diaza-bicyclo[2.2.1]heptane-2-carboxylic acid[4-(hydroxy-phenyl-methyl)-phenyl]-amide;
5-(1-o-Tolyl-ethyl)-2,5-diaza-bicyclo[2.2.1]heptane-2-carboxylic acid[1-(4-fluoro-phenyl)-ethyl]-amide;
5-(1-Methyl-2-o-tolyl-ethyl)-2,5-diaza-bicyclo[2.2.1]heptane-2-carboxylic acid[1-(4-fluoro-phenyl)-ethyl]-amide;
5-[1-(4-Fluoro-2-methyl-phenyl)-ethyl]-2,5-diaza-bicyclo[2.2.1]heptane-2-carboxylic acid(4-benzoyl-phenyl)-amide;
5-[1-(4-Fluoro-2-methyl-phenyl)-ethyl]-2,5-diaza-bicyclo[2.2.1]heptane-2-carboxylic acid(4-benzoyl-phenyl)-amide;
5-[1-(2-Chloro-phenyl)-ethyl]-2,5-diaza-bicyclo[2.2.1]heptane-2-carboxylic acid[4-(4-fluoro-phenoxy)-phenyl]-amide;
5-(1-o-Tolyl-ethyl)-2,5-diaza-bicyclo[2.2.1]heptane-2-carboxylic acid[4-(4-fluoro-phenoxy)-phenyl]-amide;
5-[1-(4-Fluoro-2-methyl-phenyl)-ethyl]-2,5-diaza-bicyclo[2.2.1]heptane-2-carboxylic acid[4-(4-fluoro-phenoxy)-phenyl]-amide;
5-[1-(2-Chloro-phenyl)-ethyl]-2,5-diaza-bicyclo[2.2.1]heptane-2-carboxylic acid[4-(4-fluoro-phenoxy)-phenyl]-amide;
5-(1,2,3,4-Tetrahydro-naphthalen-2-yl)-2,5-diaza-bicyclo[2.2.1]heptane-2-carboxylic acid(4-trifluoromethyl-phenyl)-amide;
5-(1-o-Tolyl-ethyl)-2,5-diaza-bicyclo[2.2.1]heptane-2-carboxylic acid[4-(4-fluoro-phenoxy)-phenyl]-amide fumarate salt;
5-[2-(2-Fluoro-phenyl)-1-methyl-ethyl]-2,5-diaza-bicyclo[2.2.1]heptane-2-carboxylic acid(4-trifluoromethyl-phenyl)-amide;
5-[2-(4-Fluoro-phenyl)-1-methyl-ethyl]-2,5-diaza-bicyclo[2.2.1]heptane-2-carboxylic acid(4-trifluoromethyl-phenyl)-amide;
5-(2-Cyano-1-methyl-2-phenyl-ethyl)-2,5-diaza-bicyclo[2.2.1]heptane-2-carboxylic acid(4-trifluoromethyl-phenyl)-amide;
5-[2-(2-Chloro-phenyl)-1-methyl-ethyl]-2,5-diaza-bicyclo[2.2.1]heptane-2-carboxylic acid(4-trifluoromethyl-phenyl)-amide;
5-[2-(4-Chloro-phenyl)-1-methyl-ethyl]-2,5-diaza-bicyclo[2.2.1]heptane-2-carboxylic acid(4-trifluoromethyl-phenyl)-amide;
5-[2-(2,4-Dichloro-phenyl)-1-methyl-ethyl]-2,5-diaza-bicyclo[2.2.1]heptane-2-carboxylic acid(4-trifluoromethyl-phenyl)-amide;
5-[2-(2-Chloro-6-fluoro-phenyl)-1-methyl-ethyl]-2,5-diaza-bicyclo[2.2.1]heptane-2-carboxylic acid(4-trifluoromethyl-phenyl)-amide;
5-[2-(2-Methoxy-phenyl)-1-methyl-ethyl]-2,5-diaza-bicyclo[2.2.1]heptane-2-carboxylic acid(4-trifluoromethyl-phenyl)-amide;
7-Benzyl-2,7-diaza-spiro[4.4]nonane-2-carboxylic acid(4-phenoxy-phenyl)-amide;
5-Benzyl-hexahydro-pyrrolo[3,4-c]pyrrole-2-carboxylic acid(4-phenoxy-phenyl)-amide;
5-[1-(4-Cyano-phenyl)-ethyl]-2,5-diaza-bicyclo[2.2.1]heptane-2-carboxylic acid[4-(4-fluoro-phenoxy)-phenyl]-amide;
5-[1-(4-Cyano-phenyl)-ethyl]-2,5-diaza-bicyclo[2.2.1]heptane-2-carboxylic acid[4-(4-fluoro-phenoxy)-phenyl]-amide;
5-(5-Fluoro-indan-1-yl)-2,5-diaza-bicyclo[2.2.1]heptane-2-carboxylic acid[4-(4-fluoro-phenoxy)-phenyl]-amide;
5-(5-Fluoro-indan-1-yl)-2,5-diaza-bicyclo[2.2.1]heptane-2-carboxylic acid[4-(4-fluoro-phenoxy)-phenyl]-amide;
5-(5-Fluoro-indan-1-yl)-2,5-diaza-bicyclo[2.2.1]heptane-2-carboxylic acid(4-trifluoromethyl-phenyl)-amide;
5-(5-Fluoro-indan-1-yl)-2,5-diaza-bicyclo[2.2.1]heptane-2-carboxylic acid(4-trifluoromethyl-phenyl)-amide;
5-(1,2,3,4-Tetrahydro-naphthalen-2-yl)-2,5-diaza-bicyclo[2.2.1]heptane-2-carboxylic acid[4-(4-fluoro-phenoxy)-phenyl]-amide;
5-Benzyl-hexahydro-pyrrolo[3,4-c]pyrrole-2-carboxylic acid[4-(4-fluoro-phenoxy)-phenyl]-amide;
5-Indan-2-yl-2,5-diaza-bicyclo[2.2.1]heptane-2-carboxylic acid(4-trifluoromethyl-phenyl)-amide;
5-(6-Fluoro-1,2,3,4-tetrahydro-naphthalen-2-yl)-2,5-diaza-bicyclo[2.2.1]heptane-2-carboxylic acid(4-trifluoromethyl-phenyl)-amide;
5-(6-Fluoro-1,2,3,4-tetrahydro-naphthalen-2-yl)-2,5-diaza-bicyclo[2.2.1]heptane-2-carboxylic acid[4-(4-fluoro-phenoxy)-phenyl]-amide;
7-Benzyl-2,7-diaza-spiro[4.4]nonane-2-carboxylic acid[4-(4-fluoro-phenoxy)-phenyl]-amide;
7-Indan-2-yl-2,7-diaza-spiro[4.4]nonane-2-carboxylic acid[4-(4-fluoro-phenoxy)-phenyl]-amide;
5-Indan-2-yl-2,5-diaza-bicyclo[2.2.1]heptane-2-carboxylic acid(4-phenoxy-phenyl)-amide;
5-Indan-2-yl-2,5-diaza-bicyclo[2.2.1]heptane-2-carboxylic acid[4-(4-fluoro-phenoxy)-phenyl]-amide;
5-[1-(2-Chloro-phenyl)-ethyl]hexahydro-pyrrolo[3,4-c]pyrrole-2-carboxylic acid[4-(4-fluoro-phenoxy)-phenyl]-amide;
5-[1-(2-Chloro-phenyl)-ethyl]-2,5-diaza-bicyclo[2.2.1]heptane-2-carboxylic acid[4-(3-fluoro-phenoxy)-phenyl]-amide;
5-[1-(2-Chloro-phenyl)-ethyl]-2,5-diaza-bicyclo[2.2.1]heptane-2-carboxylic acid[4-(2-fluoro-phenoxy)-phenyl]-amide;
5-[1-(2-Chloro-phenyl)-ethyl]-2,5-diaza-bicyclo[2.2.1]heptane-2-carboxylic acid[3-fluoro-4-(4-fluoro-phenoxy)-phenyl]-amide;
7-[1-(2-Chloro-phenyl)-ethyl]-2,7-diaza-spiro[4.4]nonane-2-carboxylic acid[4-O-fluoro-phenoxy)-phenyl]-amide;
5-Indan-2-yl-hexahydro-pyrrolo[3,4-c]pyrrole-2-carboxylic acid[4-(4-fluoro-phenoxy)-phenyl]-amide;
5-Indan-2-yl-2,5-diaza-bicyclo[2.2.1]heptane-2-carboxylic acid[4-(2-fluoro-phenoxy)-phenyl]-amide;
5-Indan-2-yl-2,5-diaza-bicyclo[2.2.1]heptane-2-carboxylic acid[4-(3-fluoro-phenoxy)-phenyl]-amide;
5-[1-(2-Chloro-phenyl)-ethyl]-2,5-diaza-bicyclo[2.2.1]heptane-2-carboxylic acid[4-(4-fluoro-phenoxy)-phenyl]-methyl-amide;
5-[1-(2,4-Dimethyl-phenyl)-ethyl]-2,5-diaza-bicyclo[2.2.1]heptane-2-carboxylic acid[4-(4-fluoro-phenoxy)-phenyl]-amide;
5-[1-(2,4-Dimethyl-phenyl)-ethyl]-2,5-diaza-bicyclo[2.2.1]heptane-2-carboxylic acid[4-(4-fluoro-phenoxy)-phenyl]-amide;
5-[1-(2,4-Dichloro-phenyl)-ethyl]-2,5-diaza-bicyclo[2.2.1]heptane-2-carboxylic acid[4-(4-fluoro-phenoxy)-phenyl]-amide;
5-[1-(2,4-Dichloro-phenyl)-ethyl]-2,5-diaza-bicyclo[2.2.1]heptane-2-carboxylic acid[4-(4-fluoro-phenoxy)-phenyl]-amide;
5-[1-(2,4-Di chloro-phenyl)-ethyl]-2,5-diaza-bicyclo[2.2.1]heptane-2-carb oxylic acid(4-trifluoromethyl-phenyl)-amide;
5-[1-(2,4-Dichloro-phenyl)-ethyl]-2,5-diaza-bicyclo[2.2.1]heptane-2-carboxylic acid[4-(4-chloro-phenoxy)-phenyl]-amide;
5-[1-(2,4-Dichloro-phenyl)-ethyl]-2,5-diaza-bicyclo[2.2.1]heptane-2-carboxylic acid[4-(4-chloro-phenoxy)-phenyl]-amide;
5-[1-(2,4-Dichloro-phenyl)-ethyl]-2,5-diaza-bicyclo[2.2.1]heptane-2-carboxylic acid(4-p-tolyloxy-phenyl)-amide;
5-(5-Fluoro-indan-2-yl)-2,5-diaza-bicyclo[2.2.1]heptane-2-carboxylic acid(4-benzyl-phenyl)-amide;
5-(5-Fluoro-indan-2-yl)-2,5-diaza-bicyclo[2.2.1]heptane-2-carboxylic acid(4-phenoxy-phenyl)-amide;
5-[1-(2,4-Di chloro-phenyl)-ethyl]-2,5-diaza-bicyclo[2.2.1]heptane-2-carboxylic acid(4-trifluoromethyl-phenyl)-amide;
5-[1-(2,4-Dichloro-phenyl)-ethyl]-2,5-diaza-bicyclo[2.2.1]heptane-2-carboxylic acid(4-p-tolyloxy-phenyl)-amide;
5-[1-(2-Chloro-phenyl)-ethyl]-2,5-diaza-bicyclo[2.2.1]heptane-2-carboxylic acid[4-(4-chloro-phenoxy)-phenyl]-amide;
5-[1-(2-Chloro-phenyl)-ethyl]-2,5-diaza-bicyclo[2.2.1]heptane-2-carboxylic acid[4-(4-chloro-phenoxy)-phenyl]-amide;
5-(5-Fluoro-indan-2-yl)-2,5-diaza-bicyclo[2.2.1]heptane-2-carboxylic acid(4-trifluoromethyl-phenyl)-amide;
5-(5-Fluoro-indan-2-yl)-2,5-diaza-bicyclo[2.2.1]heptane-2-carboxylic acid[4-(4-fluoro-phenoxy)-phenyl]-amide;
5-[1-(2-Chloro-4-fluoro-phenyl)-ethyl]-2,5-diaza-bicyclo[2.2.1]heptane-2-carboxylic acid[4-(4-fluoro-phenoxy)-phenyl]amide;
5-[1-(2-Chloro-4-fluoro-phenyl)-ethyl]-2,5-diaza-bicyclo[2.2.1]heptane-2-carboxylic acid[4-(4-fluoro-phenoxy)-phenyl]-amide;
1-[5-(5-Fluoro-indan-2-yl)-2,5-diaza-bicyclo[2.2.1]hept-2-yl]-2-(4-phenoxy-phenyl)-ethanone;
1-{5-[1-(2-Chloro-phenyl)-ethyl]-2,5-diaza-bicyclo[2.2.1]hept-2-yl}-2-[4-(4-fluoro-phenoxy)-phenyl]-ethanone;
1-{5-[1-(2-Chloro-phenyl)-ethyl]-2,5-diaza-bicyclo[2.2.1]hept-2-yl}-2-[4-(4-fluoro-phenoxy)-phenyl]-ethanone;
[5-(1,2,3,4-Tetrahydro-naphthalen-2-yl)-2,5-diaza-bicyclo[2.2.1]hept-2-yl]-(4-trifluoromethyl-phenyl)-methanone;
[5-(7-Fluoro-1,2,3,4-tetrahydro-naphthalen-2-yl)-2,5-diaza-bicyclo[2.2.1]hept-2-yl]-(4-trifluoromethyl-phenyl)-methanone;
5-[1-(2-Chloro-phenyl)-ethyl]-2,5-diaza-bicyclo[2.2.1]heptane-2-carboxylic acid(4-p-tolyloxy-phenyl)-amide;
5-[1-(2-Chloro-phenyl)-ethyl]-2,5-diaza-bicyclo[2.2.1]heptane-2-carboxylic acid(4-p-tolyloxy-phenyl)-amide;
5-(7-Chloro-indan-1-yl)-2,5-diaza-bicyclo[2.2.1]heptane-2-carboxylic acid(4-phenoxy-phenyl)-amide;
2-[4-(4-Fluoro-phenoxy)-phenyl]-1-[5-(1,2,3,4-tetrahydro-naphthalen-2-yl)-2,5-diaza-bicyclo[2.2.1]hept-2-yl]-ethanone;
5-[1-(2-Chloro-phenyl)-ethyl]-2,5-diaza-bicyclo[2.2.1]heptane-2-carboxylic acid[4-(4-fluoro-phenoxy)-phenyl]-methyl-amide;
2-[4-(4-Fluoro-phenoxy)-phenyl]-1-[5-(6-fluoro-1,2,3,4-tetrahydro-naphthalen-2-yl)-2,5-diaza-bicyclo[2.2.1]hept-2-yl]-ethanone;
5-(5-Fluoro-indan-2-yl)-2,5-diaza-bicyclo[2.2.1]heptane-2-carboxylic acid 4-phenoxy-benzylamide;
5-(5-Fluoro-indan-2-yl)-2,5-diaza-bicyclo[2.2.1]heptane-2-carboxylic acid 4-(4-fluoro-phenoxy)-benzylamide;
1-[5-(1,2,3,4-Tetrahydro-naphthalen-2-yl)-2,5-diaza-bicyclo[2.2.1]hept-2-yl]-2-(4-trifluoromethyl-phenyl)-ethanone;
1-[5-(7-Fluoro-1,2,3,4-tetrahydro-naphthalen-2-yl)-2,5-diaza-bicyclo[2.2.1]hept-2-yl]-2-(4-trifluoromethyl-phenyl)-ethanone;
{5-[1-(2-Chloro-phenyl)-ethyl]-2,5-diaza-bicyclo[2.2.1]hept-2-yl}-(1H-indol-2-yl)-methanone;
(5-Chloro-1H-indol-2-yl)-{5-[1-(2-chloro-phenyl)-ethyl]-2,5-diaza-bicyclo[2.2.1]hept-2-yl}-methanone;
{5-[1-(2-Chloro-phenyl)-ethyl]-2,5-diaza-bicyclo[2.2.1]hept-2-yl}-[4-(4-fluoro-phenoxy)-phenyl]methanone;
{5-[1-(2-Chloro-phenyl)-ethyl]-2,5-diaza-bicyclo[2.2.1]hept-2-yl}-[4-(4-fluoro-phenoxy)-phenyl]-methanone;
1-{5-[1-(2-Bromo-phenyl)-ethyl]-2,5-diaza-bicyclo[2.2.1]hept-2-yl}-2-[4-(4-fluoro-phenoxy)-phenyl]-ethanone;
1-{5-[1-(2-Bromo-phenyl)-ethyl]-2,5-diaza-bicyclo[2.2.1]hept-2-yl}-2-[4-(4-fluoro-phenoxy)-phenyl]-ethanone;
1-{5-[1-(2,4-Dichloro-phenyl)-ethyl]-2,5-diaza-bicyclo[2.2.1]hept-2-yl}-2-[4-(4-fluoro-phenoxy)-phenyl]-ethanone;
1-{5-[1-(2,4-Dichloro-phenyl)-ethyl]-2,5-diaza-bicyclo[2.2.1]hept-2-yl}-2-[4-(4-fluoro-phenoxy)-phenyl]-ethanone;
1-{5-[1-(2,5-Dichloro-phenyl)-ethyl]-2,5-diaza-bicyclo[2.2.1]hept-2-yl}-2-[4-(4-fluoro-phenoxy)-phenyl]-ethanone;
1-{5-[1-(2,5-Dichloro-phenyl)-ethyl]-2,5-diaza-bicyclo[2.2.1]hept-2-yl}-2-[4-(4-fluoro-phenoxy)-phenyl]-ethanone;
{5-[1-(2-Bromo-phenyl)-ethyl]-2,5-diaza-bicyclo[2.2.1]hept-2-yl}-(4-phenoxy-phenyl)-methanone;
{5-[1-(2-Bromo-phenyl)-ethyl]-2,5-diaza-bicyclo[2.2.1]hept-2-yl}-(4-phenoxy-phenyl)-methanone;
1-{5-[1-(2-Chloro-4-fluoro-phenyl)-ethyl]-2,5-diazo-bicyclo-[2.2.1]hept-2-yl}-2-[4-(4-fluoro-phenoxy)-phenyl]-ethanone;
1-{5-[1-(2-Chloro-4-fluoro-phenyl)-ethyl]-2,5-diaza-bicyclo[2.2.1]hept-2-yl}-2-[4-(4-fluoro-phenoxy)-phenyl]-ethanone;
{5-[1-(2-Chloro-4-fluoro-phenyl)-ethyl]-2,5-diaza-bicyclo[2.2.1]hept-2-yl}-[4-(4-fluoro-phenoxy)-phenyl]-methanone;
{5-[1-(2-Chloro-4-fluoro-phenyl)-ethyl]-2,5-diaza-bicyclo[2.2.1]hept-2-yl}-[4-(4-fluoro-phenoxy)-phenyl]-methanone;
1-{5-[1-(2-Chloro-6-fluoro-phenyl)-ethyl]-2,5-diaza-bicyclo[2.2.1]hept-2-yl}-2-[4-(4-fluoro-phenoxy)-phenyl]-ethanone;
1-{5-[1-(2-Chloro-6-fluoro-phenyl)-ethyl]-2,5-diaza-bicyclo[2.2.1]hept-2-yl}-2-[4-(4-fluoro-phenoxy)-phenyl]ethanone;
{5-[1-(2-Chloro-6-fluoro-phenyl)-ethyl]-2,5-diaza-bicyclo[2.2.1]hept-2-yl}-[4-(4-fluoro-phenoxy)-phenyl]-methanone;
{5-[1-(2-Chloro-6-fluoro-phenyl)-ethyl]-2,5-diaza-bicyclo[2.2.1]hept-2-yl}-[4-(4-fluoro-phenoxy)-phenyl]-methanone;
{5-[1-(2-Chloro-phenyl)-ethyl]-2,5-diaza-bicyclo[2.2.1]hept-2-yl}-[4-(3-fluoro-phenoxy)-phenyl]-methanone;
1-{5-[1-(2-Chloro-phenyl)-ethyl]-2,5-diaza-bicyclo[2.2.1]hept-2-yl}-2-[4-(3-fluoro-phenoxy)-phenyl]-ethanone;
2-[4-(3-Fluoro-phenoxy)-phenyl]-1-[5-(1-o-tolyl-ethyl)-2,5-diaza-bicyclo[2.2.1]hept-2-yl]-ethanone;
2-[4-(3-Fluoro-phenoxy)-phenyl]-1-[5-(1-o-tolyl-ethyl)-2,5-diaza-bicyclo[2.2.1]hept-2-yl]-ethanone;
3-(4-{5-[1-(2-Chloro-phenyl)-ethyl]-2,5-diaza-bicyclo[2.2.1]heptane-2-carbonyl}-phenoxy)-benzonitrile;
3-(4-{5-[1-(2-Chloro-phenyl)-ethyl]-2,5-diaza-bicyclo[2.2.1]heptane-2-carbonyl}-phenoxy)-benzonitrile;
{5-[1-(2-Chloro-phenyl)-ethyl]-2,5-diaza-bicyclo[2.2.1]hept-2-yl}-[4-(3-fluoro-phenoxy)-phenyl]-methanone;
{5-[1-(2-Chloro-phenyl)-ethyl]-2,5-diaza-bicyclo[2.2.1]hept-2-yl}-fluoro-[4-(4-fluoro-phenoxy)-phenyl]-methanone;
{5-[1-(2-Chloro-phenyl)-ethyl]-2,5-diaza-bicyclo[2.2.1]hept-2-yl}-[3-fluoro-4-(4-fluoro-phenoxy)-phenyl]-methanone;
3-{4-[5-(1-Pyridin-2-yl-ethyl)-2,5-diaza-bicyclo[2.2.1]heptane-2-carbonyl]-phenoxy}-benzonitrile; and
[4-(4-Fluoro-phenoxy)-phenyl]-[5-(1-pyridin-2-yl-ethyl)-2,5-diaza-bicyclo[2.2.1]hept-2-yl]-methanone,

and pharmaceutically acceptable salts thereof.

As used herein, a pharmaceutically acceptable salt is a salt with a pharmaceutically acceptable acid or base. Pharmaceutically acceptable acids include both inorganic acids such as hydrochloric, sulphuric, phosphoric, diphosphoric, hydrobromic or nitric acid and organic acids such as citric, fumaric, maleic, malic, ascorbic, succinic, tartaric, benzoic, acetic, methanesulfonic, ethanesulfonic, benzenesulfonic or p-toluenesulfonic acid. Pharmaceutically acceptable bases include alkali metal (e.g. sodium or potassium) and alkali earth metal (e.g. calcium or magnesium) hydroxides and organic bases such as alkyl amines, aralkyl amines or heterocyclic amines.

The compounds of the invention can contain one or more chiral centres. For the avoidance of doubt, the chemical structures depicted herein are intended to embrace all stereoisomers of the compounds shown, including racemic and non-racemic mixtures and pure enantiomers and/or diastereoisomers.

Preferred compounds of the invention are optically active isomers. Thus, for example, preferred compounds of formula (I) containing only one chiral centre include an R enantiomer in substantially pure form, an S enantiomer in substantially pure form and enantiomeric mixtures which contain an excess of the R enantiomer or an excess of the S enantiomer.

The compounds of formula (I) may be prepared by conventional routes, for example those set out in any of Schemes 1 to 5 shown below.

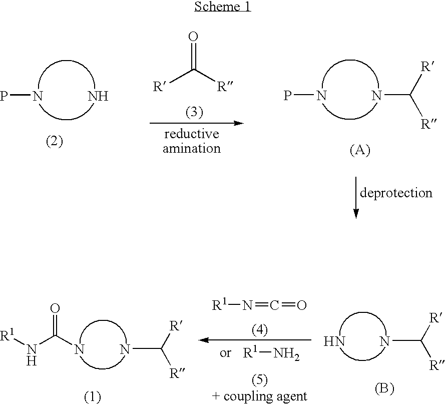

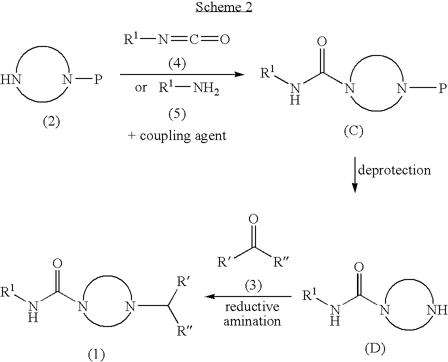

Compounds of formula (1) in which J is —NH— and —CHR′R″ is an -L-A, -L′-A′, -L-A-A′ or -L-A-L-A moiety which has a CH group α to the heterocyclic ring may be prepared utilizing standard methods, as shown in Schemes 1 and 2, from a suitably protected amine of formula (2), a carbonyl compound of formula (3) and either isocyanates of formula (4) or amines of formula (5) together with a carbonyl coupling reagent such as carbonyldiimidazole, phosgene or triphosgene. In Scheme 1 the protected amine (in which the protecting group is typically a tert-butyloxycarbonyl group) undergoes reductive amination with the carbonyl compound (3) in the presence a reducing agent such as sodium borohydride in titanium tetraisopropoxide or sodium cyanoborohydride in methanol/acetic acid to give the amine intermediate (A). Depending on the nature of groups R′ and R″ it may be possible to separate the diastereoisomers of (A) by chromatography. Deprotection of (A) under appropriate conditions generates the amine intermediate (B) which in turn is coupled under standard conditions with the isocyanate (4) or amine (5)/coupling agent to generate the product (1).

Scheme 2 shows an alternative route in which the protected amine (2) (in which the protecting group is typically a tert-butyloxycarbonyl or a benzyl group) is first converted to the urea intermediate (C) by reaction with an isocyanate (4) or amine (5)/coupling agent. The amine intermediate (D) is obtained by deprotection under appropriate conditions, and reductive amination with carbonyl compound (3) carried out as the final step to generate (1).

Compounds of formula (2), (3), (4) and (5) are either commercially available or may be prepared by standard published methods familiar to those skilled in the art.

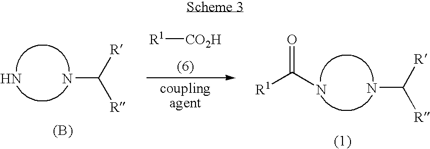

Compounds of formula (1) in which J is a direct bond and —CHR′R″ is an -L-A, -L′-A′, -L-A-A′ or -L-A-L-A moiety which has a CH group α to the heterocyclic ring may be prepared by standard methods from amine intermediates of formula (B) as described in Scheme 1 above and carboxylic acids of formula (6) by standard amide coupling methods, for example using coupling agents such as EDC/HOBT, DCC or EEDQ in the presence of a suitable solvent such as tetrahydrofuran, acetonitrile, dichloromethane or toluene.

Compounds of formulas (6) are either commercially available or may be prepared by standard published methods familiar to those skilled in the art.

In Schemes 1 to 3, the groups R′ and R″ are such that —CHR′R″ is a group -L-A, -L′-A′, -L-A-A′ or -L-A-L-A.

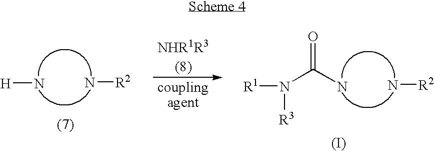

Compounds of formula (I) wherein J is —NR³— may be prepared, as shown in Scheme 4, from amines of formula (7) and amines of formula (8) together with a carbonyl coupling reagent such as carbonyldiimidazole, phosgene or triphosgene, utilising standard methods familiar to those skilled in the art such as reaction in a solvent such as tetrahydrofuran, acetonitrile, dichloromethane or toluene at a range of temperatures from ambient to reflux temperature.

Compounds of formulae (7) and (8) are either commercially available or may be prepared by standard published methods familiar to those skilled in the art.

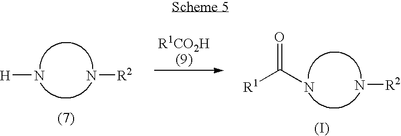

Compounds of formula (I) wherein J is a direct bond may be prepared, as shown in Scheme 5, from amines of formula (7) and carboxylic acids of formula (9) by standard amide coupling methods, for example using coupling agents such as EDC/HOBT, DCC or EEDQ in the presence of a suitable solvent such as tetrahydrofuran, acetonitrile, dichloromethane or toluene.

Compounds of formulae (7) and (9) are either commercially available or may be prepared by standard published methods familiar to those skilled in the art.

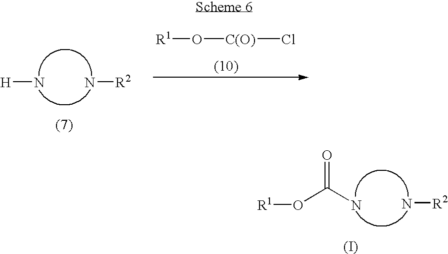

Compounds of formula (I) wherein J is —O— may be prepared, as shown in Scheme 6, from amines of formula (7) and chloroformates of formula (10) by standard amide coupling methods, for example in the presence of a base such as triethylamine, in the presence of a suitable solvent such as acetonitrile or dichloromethane.

Compounds of formulae (7) and (10) are either commercially available or may be prepared by standard published methods familiar to those skilled in the art.

The compounds of the invention are found to be inhibitors of sensory neurone specific sodium channels. The compounds of the invention are therefore therapeutically useful. The present invention also provides a compound of formula (I) or a pharmaceutically acceptable salt thereof, as defined above, for use in a method of treating the human or animal body. Such compounds are believed to be novel and the present invention also provides for these compounds.

Also provided is a pharmaceutical composition comprising a compound of the formula (I), as defined above, or a pharmaceutically acceptable salt thereof, and a pharmaceutically acceptable carrier or diluent. Said pharmaceutical composition typically contains up to 85 wt % of a compound of the invention. More typically, it contains up to 50 wt % of a compound of the invention. Preferred pharmaceutical compositions are sterile and pyrogen free. Further, the pharmaceutical compositions provided by the invention typically contain a compound of the invention which is a substantially pure optical isomer.

The compounds of the invention may be administered in a variety of dosage forms. Thus, they can be administered orally, for example as tablets, troches, lozenges, aqueous or oily suspensions, dispersible powders or granules. Preferred pharmaceutical compositions of the invention are compositions suitable for oral administration, for example tablets and capsules.

Compositions suitable for oral administration may, if required, contain a colouring or flavoring agent. Typically, a said capsule or tablet comprises from 5 to 500 mg, preferably 10 to 500 mg, more preferably 15 to 100 mg, of a compound of formula (I) or a pharmaceutically acceptable salt thereof.

The compounds of the invention may also be administered parenterally, whether subcutaneously, intravenously, intramuscularly, intrasternally, transdermally or by infusion techniques. The compounds may also be administered as suppositories.

A compound of the invention is typically formulated for administration with a pharmaceutically acceptable carrier or diluent. For example, solid oral forms may contain, together with the active compound, diluents, e.g. lactose, dextrose, saccharose, cellulose, corn starch or potato starch; lubricants, e.g. silica, talc, stearic acid, magnesium or calcium stearate, and/or polyethylene glycols; binding agents; e.g. starches, arabic gums, gelatin; methylcellulose, carboxymethylcellulose or polyvinyl pyrrolidone; disaggregating agents, e.g. starch, alginic acid, alginates or sodium starch glycolate; effervescing mixtures; dyestuffs; sweeteners; wetting agents, such as lecithin, polysorbates, laurylsulphates; and, in general, non toxic and pharmacologically inactive substances used in pharmaceutical formulations. Such pharmaceutical preparations may be manufactured in known manner, for example, by means of mixing, granulating, tableting, sugar coating, or film coating processes.

Liquid dispersions for oral administration may be syrups, emulsions and suspensions. The syrups may contain as carriers, for example, saccharose or saccharose with glycerine and/or mannitol and/or sorbitol.

Suspensions and emulsions may contain as carrier, for example a natural gum, agar, sodium alginate, pectin, methylcellulose, carboxymethylcellulose, or polyvinyl alcohol. The suspension or solutions for intramuscular injections may contain, together with the active compound, a pharmaceutically acceptable carrier, e.g. sterile water, olive oil, ethyl oleate, glycols, e.g. propylene glycol, and if desired, a suitable amount of lidocaine hydrochloride.

Solutions for injection or infusion may contain as carrier, for example, sterile water or preferably they may be in the form of sterile, aqueous, isotonic saline solutions.

The compounds of the present invention are therapeutically useful in the treatment or prophylaxis of conditions involving sodium ion flux through a sensory neurone specific (SNS) channel of a sensory neurone. Said condition may be one of hypersensitivity for example resulting from a concentration of SNS channels at the site of nerve injury or in axons following nerve injury, or may be sensitisation of the neurone for example at sites of inflammation as a result of inflammatory mediators.

Said compounds of the invention are therefore most preferred for their use in the treatment or prophylaxis of any condition involving hypersensitivity or sensitisation of a sensory neurone specific (SNS) channel of a sensory neurone.

Accordingly, the present invention also provides the use of a compound of the formula (I), or a pharmaceutically acceptable salt thereof, in the manufacture of a medicament for use in the treatment or prophylaxis of a condition involving sodium ion flux through a sensory neurone specific (SNS) channel of a sensory neurone, more specifically hypersensitivity of a sensory neurone or sensitisation of a sensory neurone specific (SNS) channel of a sensory neurone. Also provided is a method of treating a patient suffering from or susceptible to a condition involving sodium ion flux through a sensory neurone specific (SNS) channel of a sensory neurone, more specifically hypersensitivity of a sensory neurone or sensitisation of a sensory neurone specific (SNS) channel of a sensory neurone, which method comprises administering to said patient an effective amount of a compound of formula (I), or a pharmaceutically acceptable salt thereof.

The term treatment in this context is deemed to cover any effect from a cure of said condition to alleviation of any or all of the symptoms. The compounds of the invention may, where appropriate, be used prophylactically to reduce the incidence or severity of said conditions.

Specific conditions in which SNS channels are present and believed to be involved include pain, for example chronic and acute pain, hypersensitivity disorders such as bladder dysfunction and bowel disorders which may or may not also have associated pain, and demyelinating diseases.

SNS sodium channels are known to mediate pain transmission. Typically, the compounds of the invention are therefore used as analgesic agents. SNS specific sodium channels have been identified as being particularly important in the transmission of pain signals. The compounds of the invention are accordingly particularly effective in alleviating pain. Typically, therefore, said medicament is for use in alleviating pain and said patient is suffering from or susceptible to pain. The compounds of the invention are effective in alleviating both chronic and acute pain.

Acute pain is generally understood to be a constellation of unpleasant sensory, perceptual and emotional experiences of certain associate autonomic (reflex) responses, and of psychological and behavioural reactions provoked by injury or disease. A discussion of acute pain can be found at Halpern (1984) Advances in Pain Research and Therapy, Vol. 7, p. 147. Tissue injury provokes a series of noxious stimuli which are transduced by nociceptors to impulses transmitted to the spinal cord and then to the upper part of the nervous system. Examples of acute pains which can be alleviated with the compounds of the invention include musculoskeletal pain, for example joint pain, lower back pain and neck pain, dental pain, post-operative pain, obstetric pain, for example labour pain, acute headache, neuralgia, myalgia, and visceral pain.

Chronic pain is generally understood to be pain that persists beyond the usual course of an acute disease or beyond a reasonable time for an injury to heal. A discussion of chronic pain can be found in the Halpern reference given above. Chronic pain is sometimes a result of persistent dysfunction of the nociceptive pain system. Examples of chronic pains which can be alleviated with the compounds of the invention include trigeminal neuralgia, post-herpetic neuralgia (a form of chronic pain accompanied by skin changes in a dermatomal distribution following damage by acute Herpes Zoster disease), diabetic neuropathy, causalgia, “phantom limb” pain, pain associated with osteoarthritis, pain associated with rheumatoid arthritis, pain associated with cancer, pain associated with HIV, neuropathic pain, migraine and other conditions associated with chronic cephalic pain, primary and secondary hyperalgesia, inflammatory pain, nociceptive pain, tabes dorsalis, spinal cord injury pain, central pain, post-herpetic pain, noncardiac chest pain, irritable bowel syndrome and pain associated with bowel disorders and dyspepsia.

Some of the chronic pains set out above, for example, trigeminal neuralgia, diabetic neuropathic pain, causalgia, phantom limb pain and central post-stroke pain, have also been classified as neurogenic pain. One non-limiting definition of neurogenic pain is pain caused by dysfunction of the peripheral or central nervous system in the absence of nociceptor stimulation by trauma or disease. The compounds of the invention can, of course, be used to alleviate or reduce the incidence of neurogenic pain

Examples of bowel disorders which can be treated or prevented with the compounds of the invention include inflammatory bowel syndrome and inflammatory bowel disease, for example Crohn's disease and ulcerative colitis.

Examples of bladder dysfunctions which can be treated or prevented with the compounds of the invention include bladder hyper reflexia and bladder inflammation, for example interstitial cystitis, overactive (or unstable) bladder (OAB), more specifically urinary incontinence, urgency, frequency, urge incontinence and nocturia. The compounds of the invention can also be used to alleviate pain associated with bladder hyper reflexia or bladder inflammation.

Examples of demyelinating diseases which can be treated or prevented with the compounds of the invention are those in which SNS channels are known to be expressed by the demyelinated neurones and which may or may not also have associated pain. A specific example of such a demyelinating disease is multiple sclerosis. The compounds of the invention can also be used to alleviate pain associated with demyelinating diseases such as multiple sclerosis.

The compounds of the invention have additional properties as they are capable of inhibiting voltage dependent sodium channels. They can therefore be used, for example, to protect cells against damage or disorders which results from overstimulation of sodium channels.

The compounds of the invention are useful in the treatment and prevention of peripheral and central nervous system disorders. They can therefore additionally be used in the treatment or prevention of an affective disorder, an anxiety disorder, a behavioural disorder, a cardiovascular disorder, a central or peripheral nervous system degenerative disorder, a central nervous system injury, a cerebral ischaemia, a chemical injury or substance abuse disorder, a cognitive disorder, an eating disorder, an eye disease, Parkinson's disease or a seizure disorder.

Examples of affective disorders which can be treated or prevented with the compounds of the invention include mood disorders, bipolar disorders (both Type 1 and Type II) such as seasonal affective disorder, depression, manic depression, atypical depression and monodepressive disease, schizophrenia, psychotic disorders, mania and paranoia.

Examples of anxiety disorders which can be treated or prevented with the compounds of the invention include generalised anxiety disorder (GAD), panic disorder, panic disorder with agoraphobia, simple (specific) phobias (e.g. arachnophobia, performance anxiety such as public speaking), social phobias, post-traumatic stress disorder, anxiety associated with depression, and obsessive compulsive disorder (OCD).

Examples of behavioural disorders which can be treated or prevented with the compounds of the invention include behavioural and psychological signs and symptoms of dementia, age-related behavioural disorders, pervasive development disorders such as autism, Asperger's Syndrome, Retts syndrome and disintegrative disorder, attention deficit disorder, aggressivity, impulse control disorders and personality disorder.

Examples of cardiovascular disorders which can be treated or prevented with the compounds of the invention include cardiac arrthymia, atherosclerosis, cardiac arrest, thrombosis, complications arising from coronary artery bypass surgery, myocardial infarction, reperfusion injury, intermittant claudication, ischaemic retinopathy, angina, pre-eclampsia, hypertension, congestive cardiac failure, restenosis following angioplasty, sepsis and septic shock.

Examples of central and peripheral nervous system degenerative disorders which can be treated or prevented with the compounds of the invention include corticobasal degeneration, disseminated sclerosis, Freidrich's ataxia, motorneurone diseases such as amyotrophic lateral sclerosis and progressive bulbar atrophy, multiple system atrophy, myelopathy, radiculopathy, peripheral neuropathies such as diabetic neuropathy, tabes dorsalis, drug-induced neuropathy and vitamin deficiency, systemic lupus erythamatosis, granulomatous disease, olivo-ponto-cerebellar atrophy, progressive pallidal atrophy, progressive supranuclear palsy and spasticity.

Examples of central nervous system injuries which can be treated with the compounds of the invention include traumatic brain injury, neurosurgery (surgical trauma), neuroprotection for head injuries, raised intracranial pressure, cerebral oedema, hydrocephalus and spinal cord injury.

Examples of cerebral ischaemias which can be treated or prevented with the compounds of the invention include transient ischaemic attack, stroke, for example thrombotic stroke, ischaemic stroke, embolic stroke, haemorrhagic stroke or lacunar stroke, subarachnoid haemorrhage, cerebral vasospasm, peri-natal asphyxia, drowning, cardiac arrest and subdural haematoma.

Examples of chemical injuries and substance abuse disorders which can be treated or prevented with the compounds of the invention include drug dependence, for example opiate dependence, benzodiazepine addition, amphetamine addiction and cocaine addiction, alcohol dependence, methanol toxicity, carbon monoxide poisoning and butane inhalation.

Examples of cognitive disorders which can be treated or prevented with the compounds of the invention include dementia, Alzheimer Disease, Frontotemporal dementia, multi-infarct dementia, AIDS dementia, dementia associated with Huntingtons Disease, Lewy body Dementia, Senile dementia, age-related memory impairment, cognitive impairment associated with dementia, Korsakoff syndrome and dementia pugilans.

Examples of eating disorders which can be treated or prevented with the compounds of the invention include anorexia nervosa, bulimia, Prader-Willi syndrome and obesity.

Examples of eye diseases which can be treated or prevented with the compounds of the invention include drug-induced optic neuritis, cataract, diabetic neuropathy, ischaemic retinopathy, retinal haemorrhage, retinitis pigmentosa, acute glaucoma, in particular acute normal tension glaucoma, chronic glaucoma, in particular chronic normal tension glaucoma, macular degeneration, retinal artery occlusion and retinitis.

Examples of Parkinson's diseases which can be treated or prevented with the compounds of the invention include drug-induced Parkinsonism, post-encephalitic Parkinsonism, Parkinsonism induced by poisoning (for example MPTP, manganese or carbon monoxide poisoning), Dopa-responsive dystonia-Parkinsonism, posttraumatic Parkinson's disease (punch-drunk syndrome), Parkinson's with on-off syndrome, Parkinson's with freezing (end of dose deterioration) and Parkinson's with prominent dyskinesias.

Examples of seizure disorders which can be treated or prevented with the compounds of the invention include epilepsy and post-traumatic epilepsy, partial epilepsy (simple partial seizures, complex partial seizures, and partial seizures secondarily generalised seizures), generalised seizures, including generalised tonicclonic seizures (grand mal), absence seizures (petit mal), myoclonic seizures, atonic seizures, clonic seizures, and tonic seizures, Lennox Gastaut, West Syndrome (infantile spasms), multiresistant seizures and seizure prophylaxis (antiepileptogenic).

The compounds of the present invention are also useful in the treatment and prevention of tinnitus.

A therapeutically effective amount of a compound of the invention is administered to a patient. A typical dose is from about 0.001 to 50 mg per kg of body weight, for example 0.01 to 10 mg, according to the activity of the specific compound, the age, weight and conditions of the subject to be treated, the type and severity of the disease and the frequency and route of administration. Preferably, daily dosage levels are from 5 mg to 2 g.

The following Examples illustrate the invention. They do not, however, limit the invention in any way. In this regard, it is important to understand that the particular assays used in the Examples section are designed only to provide an indication of activity in inhibiting SNS specific sodium channels. A negative result in any one particular assay is not determinative.

### EXAMPLES

### Example 82

### 5-[2-(2-Chloro-phenyl)-1-methyl-ethyl]-2,5-diaza-bicyclo[2.2.1]heptane-2-carboxylic acid(4-trifluoromethyl-phenyl)-amide (Method A)

(i) 1-Isocyanato-4-trifluoromethyl-benzene (990 mg, 5.29 mmol) was added to a stirred solution of 2,5-diaza-bicyclo[2.2.1]heptane-2-carboxylic acid tert-butyl ester (1 g, 5.04 mmol) in dichloromethane (DCM) (20 ml) and stirred overnight at room temperature. Trifluoroacetic acid (4 ml) was added and stirred for 2 h. The solution was made basic with 2N sodium hydroxide solution, separated, extracted with DCM and the combined organic extracts dried and evaporated. The residue was loaded onto a flash silica column, eluted with ethyl acetate to remove impurities followed by methanol/DCM (1:9) to elute the product. Evaporation gave 2,5-Diaza-bicyclo[2.2.1]heptane-2-carboxylic acid(4-trifluoromethyl-phenyl)-amide (905 mg) as a brown solid.

(ii) Sodium cyanoborohydride (30 mg, 0.47 mmol) was added to a stirred solution of 1-(2-chloro-phenyl)-propan-2-one (54 mg, 0.32 mmol) and the product of step (i) (100 mg, 0.35 mmol) in a 1% mixture of acetic acid in methanol (5 ml). The mixture was heated under reflux overnight, then evaporated and the residue purified by flash chromatography (silica gel, DCM/methanol) giving the title compound (97 mg).

Other compounds prepared by Method A as described for Example 82 using the appropriate starting materials are listed in the TABLE.

### Example 76

### 5-[1-(2-Chloro-phenyl)-ethyl]-2,5-diaza-bicyclo[2.2.1]heptane-2-carboxylic acid[4-(4-fluoro-phenoxy)-phenyl]amide (Method B)

(i) Titanium tetraisopropoxide (4 ml) was added drop wise with stirring to 1-(2-chloro-phenyl)-ethanone (1.36 ml, 10.5 mmol). 2,5-Diaza-bicyclo[2.2.1]heptane-2-carboxylic acid tert-butyl ester (2.01 g, 10.1 mmol) was added in portions and stirred overnight. The mixture was diluted with ethanol (40 ml), sodium borohydride (1.32 g) added and stirred for 1 h before being poured into DCM and water added with stirring. Filtration, extraction of the aqueous phase, drying and evaporation gave a mixture of diastereoisomers of 5-[1-(2-chloro-phenyl)-ethyl]-2,5-diaza-bicyclo[2.2.1]heptane-2-carboxylic acid tert-butyl ester (3.56 g). The individual diastereoisomers were separated by chromatography (silica gel, ethyl acetate/hexane 1:1).

(ii) The higher R_{F} diastereoisomer (885 mg, 2.63 mmol) was stirred at 0° C. with neat trifluoroacetic acid (12 ml) for 1 h, diluted with DCM (60 ml) and water (20 ml) and adjusted to pH9 with 35% aq. ammonia solution at 0° C. The organic layer was separated, dried and evaporated giving 2-[1-(2-chloro-phenyl)-ethyl]-2,5-diaza-bicyclo[2.2.1]heptane (462 mg).

(iii) A solution of 4-(4-fluoro-phenoxy)-phenylamine (536.5 mg, 2.64 mmol) in dichloromethane (11 ml) was added drop wise over 1.5 h to carbonyldiimidazole (CDI) and stirred for 5.25 h. The intermediate from ii) above (312.5 mg) in dry DCM (5.5 ml) was added and the mixture stirred overnight. Evaporation followed by chromatography gave the title compound (247.3 mg) as a pale yellow foam.

Other compounds prepared by Method B as described for Example 76 using the appropriate starting materials are listed in the TABLE.

### Example 12

### 5-[1-(2-Chloro-phenyl)-ethyl]-2,5-diaza-bicyclo[2.2.1]heptane-2-carboxylic acid(4-phenoxy-phenyl)-amide (Method C)

(iii) 1-Isocyanato-4-phenoxy-benzene (64 mg, 0.31 mmol) in acetonitrile (2 ml) was added to 2-[1-(2-chloro-phenyl)-ethyl]-2,5-diaza-bicyclo[2.2.1]heptane (see Example 76 step (ii)) (60 mg, 0.24 mmol) in acetonitrile (2 ml) and stirred for 2 h. The solvent was removed in vacuo and the residue purified by chromatography (silica gel, ethyl acetate/hexane 1:1) giving the title compound (14.6 mg) as a clear glass.

Other compounds prepared by Method C as described for Example 12 using the appropriate starting materials are listed in the TABLE.

### Example 115

### 5-[1-(2-Chloro-phenyl)-ethyl]-2,5-diaza-bicyclo[2.2.1]heptane-2-carboxylic acid[4-(4-fluoro-phenoxy)-phenyl]-methyl-amide (Method D)

(iii) [4-(4-Fluoro-phenoxy)-phenyl]-methylamine (83 mg, 0.38 mmol) was dissolved in DCM (10 ml) and a 20% solution of phosgene in toluene (190 μl, 0.38 mmol) added and stirred for 1 h at room temperature. A solution of 2-[1-(2-chloro-phenyl)-ethyl]-2,5-diaza-bicyclo[2.2.1]heptane (prepared from the lower R_{F} diastereoisomer of 5-[1-(2-chloro-phenyl)-ethyl]-2,5-di aza-bicyclo[2.2.1]heptane-2-carboxylic acid tert-butyl ester using the method of Example 76 step (ii)) in DCM (1 ml) was added, stirred for 2 h then triethylamine (107 μl, 0.76 mmol) added and stirred overnight at room temperature. The reaction was quenched with 20% sodium hydroxide solution and the organic layer washed with 5% citric acid and brine then dried and evaporated. The residue was purified by chromatography (silica gel, ethyl acetate/hexane 9:1) giving the title compound (80 mg).

Other compounds prepared by Method D as described for Example 115 using the appropriate starting materials are listed in the TABLE.

### Example 89

### 5-Benzyl-hexahydro-pyrrolo[3,4-c]pyrrole-2-carboxylic acid(4-phenoxy-phenyl)-amide (Method E)

Triethylamine (117 μl, 0.83 mmol) was added to a stirred suspension of 2-benzyl-octahydro-pyrrolo[3,4-c]pyrrole dihydrochloride (109 mg, 0.39 mmol) in DCM and stirred until dissolved. 1-Isocyanato-4-phenoxy-benzene (92 mg, 0.43 mmol) in DCM (2 ml) was added and stirred for 2 h. The solvent was evaporated in vacuo and the residue purified by flash chromatography (silica gel, DCM/methanol 19:1) giving the title compound (130 mg).

Other compounds prepared by Method E as described for Example 89 using the appropriate starting materials are listed in the TABLE.

### Example 103

### 7-Benzyl-2,7-diaza-spiro[4.4]nonane-2-carboxylic acid[4-(4-fluoro-phenoxy)-phenyl]-amide and

### Example 104

### 7-Indan-2-yl-2,7-diaza-spiro[4.4]nonane-2-carboxylic acid[4-(4-fluoro-phenoxy)-phenyl]-amide (Method F)

(i) Carbonyldiimidazole (757 mg, 4.67 mmol) was dissolved in DCM (40 ml) and warmed to 30° C. 4-(4-Fluoro-phenoxy)-phenylamine (947 mg, 4.67 mmol) in DCM (10 ml) was added and the mixture stirred for 1 h. A solution of 2-benzyl-2,7-diaza-spiro[4.4]nonane dihydrochloride (900 mg, 3.1 mmol) in DCM (10 ml) and triethylamine (875 μl, 6.2 mmol) was added and the mixture stirred for 65 h. The solvent was evaporated in vacuo and the residue stirred in methanol (50 mol) for 30 min. Insoluble material was filtered off and the filtrate evaporated in vacuo. The residue was purified by flash chromatography giving 7-benzyl-2,7-diaza-spiro[4.4]nonane-2-carboxylic acid[4-(4-fluoro-phenoxy)-phenyl]amide (Example 103) (824 mg).

(ii) The product from step (i) (380 mg) was dissolved in methanol (25 ml), palladium hydroxide was added and the mixture hydrogenated at 3 mbar for 18 h. Purification by flash chromatography (silica gel, DCM/methanol/ammonia gradient from 90:10:0 to 90:10:1) gave 2,7-Diaza-spiro[4.4]nonane-2-carboxylic acid[4-(4-fluoro-phenoxy)-phenyl]amide (160 mg).

(iii) The product from step (ii) (80 mg, 0.22 mmol) was dissolved in 1% acetic acid in methanol (10 ml). Indan-2-one (38 mg, 0.29 mmol) was added followed by sodium cyanoborohydride (29 mg, 0.45 mmol) and then heated under reflux for 6 h. The reaction mixture was evaporated and the residue purified by flash chromatography (silica gel, DCM/methanol gradient from 100:0 to 95:5) giving 7-Indan-2-yl-2,7-diaza-spiro[4.4]nonane-2-carboxylic acid[4-(4-fluoro-phenoxy)phenyl]-amide (Example 104) (18.2 mg).

Other compounds prepared by Method F as described for Examples 103 and 104 using the appropriate starting materials are listed in the TABLE.

### Example 36

### 2-(4-Phenoxy-phenyl)-1-[5-(1-o-tolyl-ethyl)-2,5-diaza-bicyclo[2.2.1]hept-2-yl]-ethanone (Method G)

(i) 2,5-Diaza-bicyclo[2.2.1]heptane-2-carboxylic acid tert-butyl ester (2.07 g, 10.4 mmol) was added portionwise with stirring to titanium tetraisopropoxide (6.0 ml, 20.2 mmol). 1-o-Tolyl-ethanone (1.37 ml, 10.4 mmol) was added and the reaction mixture stirred for 6 h. Ethanol (50 ml) was added followed by sodium borohydride (1.22 g) and the mixture stirred overnight at room temperature. The resulting suspension was poured in to stirred DCM (500 ml) and water (50 ml) added. The organic layer was separated and the aqueous phase extracted with DCM. The organic layers were filtered through celite, dried and evaporated giving a mixture of diastereoisomers of 5-(1-o-Tolyl-ethyl)-2,5-diaza-bicyclo[2.2.1]heptane-2-carboxylic acid tert-butylamide. The individual diastereoisomers were separated by column chromatography (silica gel, ethyl acetate/DCM) giving the higher R_{F} isomer (0.73 g) and the lower R_{F} isomer (1.76 g).

(ii) The higher R_{F} isomer from step (i) (0.73 g, 2.3 mmol) was dissolved in DCM (16 ml), 4M HCl solution in 1,4-dioxane (5.9 ml, 23.8 mmol) added and the mixture stirred at room temperature for 1.5 h. The solvent was evaporated, the residue dissolved in DCM and water, basified with concentrated (0.880) ammonia solution, separated and the aqueous phase extracted with DCM. The combined organic phases were washed with brine, dried and evaporated giving 2-(1-o-tolyl-ethyl)-2,5-diaza-bicyclo[2.2.1]heptane (0.51 g).

(iii) (4-Phenoxy-phenyl)-acetic acid (67 mg, 0.29 mmol) was dissolved in DCM (2.5 ml), (3-Dimethylamino-propyl)-ethyl-carbodiimide hydrochloride (EDC) (56.2 mg, 0.29 mmol) added and stirred for 15 min. The product from step (ii) (63.3 mg, 0.29 mmol) in DCM (2 ml) was added and stirred at room temperature for 4 h. The mixture was evaporated and the residue purified by flash chromatography (silica gel, ethyl acetate) giving the title compound (85 mg).

Other compounds prepared by Method G as described for Example 36 using the appropriate starting materials are listed in the TABLE.

NMR data for some of the compounds listed in the TABLE

| Example | |
|---|---|
| Number | NMR data |
| 4 | 1H NMR (CDCl₃) δ; 1.32, 1.92 (3H, 2 × d, J = 6.4, 6.3), 1.69-2.03 (2H, m), 2.36, |
| | 2.40 (3H, 2 × s), 2.69, 4.13 (1H, 2 × d, J = 9.6, 8.5), 2.87, 3.33 (1H, 2 × dd, J = 9.9 J′ = 2.0, J = 8.4 |
| | J′ = 2.4), 3.14-3.20 (1H, m), 3.50, 3.62 (1H, 2 × d, J = 8.6, 8.1), 3.58, 3.68 (1H, br s), |
| | 3.80-3.91 (1H, m), 4.55 (1H, br s), 6.08 (1H, s), 6.96-7.03 (4H, m), 7.05-7.12 (1H, m), |
| | 7.12-7.18 (2H, m), 7.18-7.26 (2H, m), 7.30-7.44 (4H, m), 7.52, 7.56 (1H, 2 × d, J = 7.7, |
| | 8.3) |
| | (a mixture of diastereoisomers) |
| 12 | 1H NMR (CDCl₃) δ; 1.30 (3H, d, J = 6.6), 1.78 (1H, br d J = 9.9), 1.93-1.99 (1H, m), |
| | 2.66 (1H, br d, J = 10.7), 2.89 (1H, dd, J = 9.7 J′ = 2.2), 3.34 (1H, dd, J = 8.4 J′ = 2.3), |
| | 3.62 (1H, d, J = 8.3), 3.68 (1H, s), 4.15 (1H, q, J = 6.4), 4.55 (1H, s), 6.16 (1H, s), |
| | 6.95-7.02 (4H, m), 7.05-7.11 (1H, m), 7.14-7.21 (1H, m), 7.25-7.40 (6H, m), 7.64 (1H, dd, J = 7.6 |
| | J′ = 1.8) |
| 76 | 1H NMR (CDCl₃) δ; 1.30 (3H, d, J = 6.3), 1.79, (1H, br d J = 9.6), 1.97 (1H, br d, J = 9.9), |
| | 2.66 (1H, d, J = 9.8), 2.90 (1H, dd, J = 9.7 J′ = 2.2), 3.35 (1H, dd, J = 8.4 J′ = 2.3), |
| | 3.62 (1H, d, J = 8.4), 3.69 (1H, s), 4.15 (1H, q, J = 6.4), 4.55 (1H, s), 6.06 (1H, s), |
| | 6.91-7.06 (6H, m), 7.15-7.21 (1H, m), 7.25-7.31 (1H, m), 7.33-7.40 (3H, m), 7.64 (1H, dd, J = 7.9 |
| | J′ = 1.8) |
| 87 | 1H NMR (d⁶-DMSO) δ; 1.50, 1.67 (3H, 2 × d, J = 6.0, 6.7), 2.05-2.31 (2H, m), 2.32 (3H, |
| | s), 2.37, 2.41 (3H, 2 × s), 2.64-2.70 (1H, m), 3.22-4.15 (3H, m), 4.56-4.88 (3H, m), |
| | 6.90-7.04 (4H, m), 7.17-7.25 (2H, m), 7.25-7.42 (3H, m), 7.45-7.56 (2H, m), 7.58-7.65 (1H, |
| | m), 8.56 (1H, s), 9.60, 10.49 (1H, 2 × br s) |
| 171 | 1H NMR (CDCl₃) δ; 1.21 (3H, t, J = 6.3), 1.62, 1.74 (1H, 2 × d, J = 9.7, 9.2), 1.86, |
| | 1.93 (1H, 2 × d, J = 9.8, 9.5), 2.25, 2.28 (3H, 2 × s), 2.05, 2.51 (1H, 2 × d, J = 9.4, 10.2), |
| | 2.75-2.85 (1H, m), 3.26-3.33 (1H, m), 3.55-3.72 (4H, m), 3.75-3.81 (1H, m), 4.26, 4.76 (1H, 2 × s), |
| | 6.62-6.81 (3H, m), 6.92-7.02 (2H, m), 7.06-7.33 (6H, m), 7.47 (1H, t, J = 7) |
| 176 | 1H NMR (CDCl₃) δ; 1.23, 1.31 (3H, 2 × d, J = 6.3, 6.8), 1.73-2.01 (2H, m), 2.50, |
| | 2.73 (1H, 2 × d, J = 9.7, 10.1), 2.88, 2.94 (1H, 2 × dd, J = 10.1 J′ = 2.0, J = 9.6 J′ = 2.0), |
| | 3.51-3.63 (2H, m), 3.76-3.83 (1H, m), 4.06, 4.21 (1H, 2 × q, J = 6.4, 6.4), 4.25, 4.80 (1H, 2 × s), |
| | 6.89-7.09 (5H, m), 7.12-7.46 (5H, m), 7.53-7.61 (1H, m) |
| 182 | 1H NMR (d⁶-DMSO) δ; 1.57, 1.65 (3H, 2 × d, J = 7.0, 6.7), 2.04-2.40 (3H, m), 2.37 (6H, |
| | s), 2.72-3.71 (2H, m), 3.74-3.91 (2H, m), 4.08-5.04 (2H, m), 6.09-6.95 (2H, br s), |
| | 6.95-7.05 (1H, m), 7.11-7.28, 7.44-7.67 (6H, 2 × m), 7.77-7.87 (1H, m), 7.94, 7.96 (1H, 2 × d, J = 2.9, |
| | 2.6), 8.20, 8.22 (1H, 2 × d, J = 2.6, 2.6), 8.76, 8.85 (1H, 2 × s), 10.03, 10.61 (1H, 2 × s) |

### Biological Screening

Inhibition of Human Naᵥ1.8 stably expressed in SH-SY-5Y Cells

A SH-SY-5Y neuroblastoma cell line stably expressing the human Naᵥ1.8 (hNaᵥ1.8) ion channel was constructed. This cell line has been used to develop a medium to high throughput assay for determining the ability of test compounds to inhibit membrane depolarisation mediated via the hNaᵥ1.8 channel.

SH-SY-5Y hNaᵥ1.8 are grown in adherent monolayer culture using 50:50 Ham's F-12/EMEM tissue culture medium supplemented with 15% (v/v) foetal bovine serum; 2 mM L-glutamine, 1% NEAR and 600 μg·ml⁻¹ Geneticin sulphate. Cells are removed from the tissue culture flask using trypsin/EDTA and re-plated into black walled, clear bottom 96-well assay plates at 50,000 cells·well⁻¹ 24 hours prior to assay.

On the day of assay the cell assay plates are washed to remove cell culture medium using a sodium free assay buffer (145 mM tetramethyl ammonium chloride; 2 mM calcium chloride; 0.8 mM magnesium chloride hexahydrate; 10 mM HEPES; 10 mM glucose; 5 mM potassium chloride, pH 7.4). Fluorescent membrane potential dye solution (FLIPR™ membrane potential dye, Molecular Devices Corporation), containing 10 μM of a pyrethroid to prevent channel inactivation and 250 nM tetrodotoxin (TTX) to reduce interference from TTX-sensitive sodium channels present in the cell line. Test compound, initially dissolved in dimethyl sulfoxide but further diluted in sodium free buffer, is added to achieve the final test concentration range of 100 μM-0.05 μM.

Cell plates are incubated for 30 minutes at room temperature to allow equilibration of dye and test compound. Plates are then transferred to a fluorescence plate reader for fluorescence measurement using an excitation wavelength of 530 nm whilst measuring fluorescence emission at 565 nm. Baseline fluorescence levels are first determined before the addition of a sodium containing buffer (220 mM sodium chloride; 2 mM calcium chloride; 0.8 mM magnesium chloride hexahydrate; 10 mM HEPES; 10 mM glucose; 5 mM potassium chloride. pH 7.4) to cause membrane depolarisation in those cells where channel block has not been effected (final sodium concentration=72.5 mM). Membrane depolarisation is registered by an increase in fluorescence emission at 565 nm.

The change in fluorescence seen in each test well upon the addition of sodium containing buffer is calculated relative to the baseline fluorescence for that well. This figure is then used for calculating the IC₅₀ for each test compound.

TABLE
Summary of synthesis methods, characterisation data and biological activity.

| | | | | (ES+) | |
|---|---|---|---|---|---|
| Example | | | LCMS Ret.n | m/z | hNaᵥ1.8 |
| No | Compound Name | Method | time¹ | (M + H) | IC₅₀ |
| 1 | 5-(3-Methyl-thiophen-2-ylmethyl)-2,5-diaza- | C | 4.15 | 420 | 0.41 |
| | bicyclo[2.2.1]heptane-2-carboxylic acid (4- | | | | |
| | phenoxy-phenyl)-amide | | | | |
| 2 | 5-(3-Methyl-thiophen-2-ylmethyl)-2,5-diaza- | B | 3.77 | 421 | 3.74 |
| | bicyclo[2.2.1]heptane-2-carboxylic acid (6- | | | | |
| | phenoxy-pyridin-3-yl)-amide | | | | |
| 3 | 5-(3-Methyl-thiophen-2-ylmethyl)-2,5-diaza- | C | 4.20 | 400 | 1.03 |
| | bicyclo[2.2.1]heptane-2-carboxylic acid (4- | | | | |
| | butoxy-phenyl)-amide | | | | |
| 4 | 5-(1-o-Tolyl-ethyl)-2,5-diaza- | C | 4.35 | 428 | 0.32 |
| | bicyclo[2.2.1]heptane-2-carboxylic acid (4- | | | | |
| | phenoxy-phenyl)-amide | | | | |
| 5 | 5-(1-o-Tolyl-ethyl)-2,5-diaza- | C | 4.35 | 408 | 0.57 |
| | bicyclo[2.2.1]heptane-2-carboxylic acid (4- | | | | |
| | butoxy-phenyl)-amide | | | | |
| 6 | 5-(1-o-Tolyl-ethyl)-2,5-diaza- | C | 4.27 | 404 | 0.99 |
| | bicyclo[2.2.1]heptane-2-carboxylic acid (4- | | | | |
| | trifluoromethyl-phenyl)-amide | | | | |
| 7 | 5-[1-(2-Chloro-4-fluoro-phenyl)-ethyl]-2,5- | C | 4.47 | 466 | 4.32 |
| | diaza-bicyclo[2.2.1]heptane-2-carboxylic acid | | | | |
| | (4-phenoxy-phenyl)-amide | | | | |
| 8 | 5-(1-o-Tolyl-ethyl)-2,5-diaza- | C | 4.26 | 428 | 0.68 |
| | bicyclo[2.2.1]heptane-2-carboxylic acid (3- | | | | |
| | phenoxy-phenyl)-amide | | | | |
| 9 | 5-[1-(2,6-Difluoro-phenyl)-ethyl]-2,5-diaza- | C | 4.00 | 450 | 0.87 |
| | bicyclo[2.2.1]heptane-2-carboxylic acid (4- | | | | |
| | phenoxy-phenyl)-amide | | | | |
| 10 | 5-[1-(2,6-Difluoro-phenyl)-ethyl]-2,5-diaza- | C | 3.93 | 450 | 1.10 |
| | bicyclo[2.2.1]heptane-2-carboxylic acid (4- | | | | |
| | phenoxy-phenyl)-amide | | | | |
| 11 | 5-[1-(2-Chloro-phenyl)-ethyl]-2,5-diaza- | C | 4.27 | 448 | 1.80 |
| | bicyclo[2.2.1]heptane-2-carboxylic acid (4- | | | | |
| | phenoxy-phenyl)-amide | | | | |
| 12 | 5-[1-(2-Chloro-phenyl)-ethyl]-2,5-diaza- | C | 4.25 | 448 | 0.36 |
| | bicyclo[2.2.1]heptane-2-carboxylic acid (4- | | | | |
| | phenoxy-phenyl)-amide | | | | |
| 13 | 5-[1-(2,6-Difluoro-phenyl)-ethyl]-2,5-diaza- | C | 3.93 | 426 | 6.36 |
| | bicyclo[2.2.1]heptane-2-carboxylic acid (4- | | | | |
| | trifluoromethyl-phenyl)-amide | | | | |
| 14 | 5-[1-(3-Methyl-thiophen-2-yl)-ethyl]-2,5-diaza- | C | 4.06 | 434 | 0.90 |
| | bicyclo[2.2.1]heptane-2-carboxylic acid (4- | | | | |
| | phenoxy-phenyl)-amide | | | | |
| 15 | 5-[1-(3-Methyl-thiophen-2-yl)-ethyl]-2,5-diaza- | C | 4.06 | 410 | 3.04 |
| | bicyclo[2.2.1]heptane-2-carboxylic acid (4- | | | | |
| | trifluoromethyl-phenyl)-amide | | | | |
| 16 | 5-(1-o-Tolyl-ethyl)-2,5-diaza- | B | 3.63 | 468 | 25.59 |
| | bicyclo[2.2.1]heptane-2-carboxylic acid {4-[2- | | | | |
| | (2-ethoxy-ethoxy)-ethoxy]-phenyl}-amide | | | | |
| 17 | 5-(1-o-Tolyl-ethyl)-2,5-diaza- | B | 3.66 | 424 | 27.97 |
| | bicyclo[2.2.1]heptane-2-carboxylic acid [4-(2- | | | | |
| | ethoxy-ethoxy)-phenyl]-amide | | | | |
| 18 | 5-(1-o-Tolyl-ethyl)-2,5-diaza- | B | 3.44 | 454 | 30.00 |
| | bicyclo[2.2.1]heptane-2-carboxylic acid {4-[2- | | | | |
| | (2-methoxy-ethoxy)-ethoxy]-phenyl}-amide | | | | |
| 19 | 5-(1-o-Tolyl-ethyl)-2,5-diaza- | B | 3.46 | 410 | 30.00 |
| | bicyclo[2.2.1]heptane-2-carboxylic acid [4-(2- | | | | |
| | methoxy-ethoxy)-phenyl]-amide | | | | |
| 20 | 5-(1-o-Tolyl-ethyl)-2,5-diaza- | B | 3.83 | 392 | 3.42 |
| | bicyclo[2.2.1]heptane-2-carboxylic acid (4- | | | | |
| | allyloxy-phenyl)-amide | | | | |
| 21 | 5-(1-o-Tolyl-ethyl)-2,5-diaza- | B | 3.48 | 423 | 28.29 |
| | bicyclo[2.2.1]heptane-2-carboxylic acid [4-(2- | | | | |
| | dimethylamino-ethoxy)-phenyl]-amide | | | | |
| 22 | 5-(1-o-Tolyl-ethyl)-2,5-diaza- | B | 3.53 | 436 | 18.92 |
| | bicyclo[2.2.1]heptane-2-carboxylic acid [4- | | | | |
| | (tetrahydro-pyran-4-yloxy)-phenyl]-amide | | | | |
| 23 | 5-(1-o-Tolyl-ethyl)-2,5-diaza- | B | 3.22 | 463 | 30.00 |
| | bicyclo[2.2.1]heptane-2-carboxylic acid {4-[2- | | | | |
| | (2-oxo-pyrrolidin-1-yl)-ethoxy]-phenyl}-amide | | | | |
| 24 | 5-(1-o-Tolyl-ethyl)-2,5-diaza- | B | 4.19 | 420 | 0.89 |
| | bicyclo[2.2.1]heptane-2-carboxylic acid (4- | | | | |
| | cyclopentyloxy-phenyl)-amide | | | | |
| 25 | 5-(1-o-Tolyl-ethyl)-2,5-diaza- | B | 3.83 | 450 | 5.38 |
| | bicyclo[2.2.1]heptane-2-carboxylic acid [4- | | | | |
| | (tetrahydro-pyran-2-ylmethoxy)-phenyl]-amide | | | | |
| 26 | 5-(1-o-Tolyl-ethyl)-2,5-diaza- | B | 3.60 | 436 | 16.65 |
| | bicyclo[2.2.1]heptane-2-carboxylic acid [4- | | | | |
| | (tetrahydro-furan-2-ylmethoxy)-phenyl]-amide | | | | |
| 27 | 5-(1-o-Tolyl-ethyl)-2,5-diaza- | B | 3.64 | 424 | 8.48 |
| | bicyclo[2.2.1]heptane-2-carboxylic acid [4-(3- | | | | |
| | methoxy-propoxy)-phenyl]-amide | | | | |
| 28 | [2-(4-{[5-(1-o-Tolyl-ethyl)-2,5-diaza- | B | 3.60 | 479 | 4.89 |
| | bicyclo[2.2.1]heptane-2-carbonyl]-amino}- | | | | |
| | phenoxy)-ethyl]-carbamic acid allyl ester | | | | |
| 29 | 5-(1-o-Tolyl-ethyl)-2,5-diaza- | B | 4.27 | 446 | 0.34 |
| | bicyclo[2.2.1]heptane-2-carboxylic acid [4-(4- | | | | |
| | fluoro-phenoxy)-phenyl]-amide | | | | |
| 30 | 5-(1-o-Tolyl-ethyl)-2,5-diaza- | C | 4.23 | 428 | 0.32 |
| | bicyclo[2.2.1]heptane-2-carboxylic acid (4- | | | | |
| | phenoxy-phenyl)-amide | | | | |
| 31 | 5-(1-o-Tolyl-ethyl)-2,5-diaza- | C | 4.15 | 442 | 0.60 |
| | bicyclo[2.2.1]heptane-2-carboxylic acid (4- | | | | |
| | benzyloxy-phenyl)-amide | | | | |
| 32 | 5-(1-o-Tolyl-ethyl)-2,5-diaza- | C | 4.20 | 428 | 0.71 |
| | bicyclo[2.2.1]heptane-2-carboxylic acid (4- | | | | |
| | phenoxy-phenyl)-amide | | | | |
| 33 | 5-(1-o-Tolyl-ethyl)-2,5-diaza- | C | 4.15 | 442 | 0.49 |
| | bicyclo[2.2.1]heptane-2-carboxylic acid (4- | | | | |
| | benzyloxy-phenyl)-amide | | | | |
| 34 | 5-(1-o-Tolyl-ethyl)-2,5-diaza- | C | 4.31 | 426 | 0.64 |
| | bicyclo[2.2.1]heptane-2-carboxylic acid (4- | | | | |
| | benzyl-phenyl)-amide | | | | |
| 35 | 5-(1-o-Tolyl-ethyl)-2,5-diaza- | C | 4.31 | 426 | 0.59 |
| | bicyclo[2.2.1]heptane-2-carboxylic acid (4- | | | | |
| | benzyl-phenyl)-amide | | | | |
| 36 | 2-(4-Phenoxy-phenyl)-1-[5-(1-o-tolyl-ethyl)- | G | 4.33 | 427 | 0.81 |
| | 2,5-diaza-bicyclo[2.2.1]hept-2-yl]-ethanone | | | | |
| 37 | 2-(4-Phenoxy-phenyl)-1-[5-(1-o-tolyl-ethyl)- | G | 4.34 | 427 | 0.96 |
| | 2,5-diaza-bicyclo[2.2.1]hept-2-yl]-ethanone | | | | |
| 38 | 5-(1-o-Tolyl-ethyl)-2,5-diaza- | C | 4.25 | 412 | 1.44 |
| | bicyclo[2.2.1]heptane-2-carboxylic acid | | | | |
| | biphenyl-4-ylamide | | | | |
| 39 | 5-(1-o-Tolyl-ethyl)-2,5-diaza- | C | 4.26 | 412 | 1.06 |
| | bicyclo[2.2.1]heptane-2-carboxylic acid | | | | |
| | biphenyl-4-ylamide | | | | |
| 40 | 5-(1-o-Tolyl-ethyl)-2,5-diaza- | B | 4.29 | 525 | 2.01 |
| | bicyclo[2.2.1]heptane-2-carboxylic acid [4-(1- | | | | |
| | benzyl-piperidin-4-yloxy)-phenyl]-amide | | | | |
| 41 | 5-(1-o-Tolyl-ethyl)-2,5-diaza- | B | 3.70 | 449 | 30.00 |
| | bicyclo[2.2.1]heptane-2-carboxylic acid [4-(2- | | | | |
| | pyrrolidin-1-yl-ethoxy)-phenyl]-amide | | | | |
| 42 | 5-(1-o-Tolyl-ethyl)-2,5-diaza- | B | 3.65 | 477 | 2.10 |
| | bicyclo[2.2.1]heptane-2-carboxylic acid {4-[2- | | | | |
| | (4-methyl-thiazol-5-yl)-ethoxy]-phenyl}-amide | | | | |
| 43 | 5-(1-o-Tolyl-ethyl)-2,5-diaza- | B | 3.21 | 446 | 30.00 |
| | bicyclo[2.2.1]heptane-2-carboxylic acid [4-(2- | | | | |
| | imidazol-1-yl-ethoxy)-phenyl]-amide | | | | |
| 44 | 5-(1-o-Tolyl-ethyl)-2,5-diaza- | B | 3.24 | 465 | 26.78 |
| | bicyclo[2.2.1]heptane-2-carboxylic acid [4-(2- | | | | |
| | morpholin-4-yl-ethoxy)-phenyl]-amide | | | | |
| 45 | 5-(1-o-Tolyl-ethyl)-2,5-diaza- | B | 3.57 | 457 | 3.27 |
| | bicyclo[2.2.1]heptane-2-carboxylic acid [4-(2- | | | | |
| | pyridin-2-yl-ethoxy)-phenyl]-amide | | | | |
| 46 | 5-[1-(4-Fluoro-phenyl)-ethyl]-2,5-diaza- | C | 3.99 | 408 | 1.31 |
| | bicyclo[2.2.1]heptane-2-carboxylic acid (4- | | | | |
| | trifluoromethyl-phenyl)-amide | | | | |
| 47 | 5-[1-(4-Fluoro-phenyl)-ethyl]-2,5-diaza- | C | 3.98 | 408 | 2.61 |
| | bicyclo[2.2.1]heptane-2-carboxylic acid (3- | | | | |
| | trifluoromethyl-phenyl)-amide | | | | |
| 48 | 5-[1-(4-Fluoro-phenyl)-ethyl]-2,5-diaza- | C | 4.07 | 432 | 0.42 |
| | bicyclo[2.2.1]heptane-2-carboxylic acid (4- | | | | |
| | phenoxy-phenyl)-amide | | | | |
| 49 | 5-[1-(4-Fluoro-phenyl)-ethyl]-2,5-diaza- | C | 4.14 | 432 | 1.08 |
| | bicyclo[2.2.1]heptane-2-carboxylic acid (3- | | | | |
| | phenoxy-phenyl)-amide | | | | |
| 50 | 5-[1-(4-Fluoro-phenyl)-ethyl]-2,5-diaza- | C | 4.03 | 408 | 2.62 |
| | bicyclo[2.2.1]heptane-2-carboxylic acid (3- | | | | |
| | trifluoromethyl-phenyl)-amide | | | | |
| 51 | 5-[1-(4-Fluoro-phenyl)-ethyl]-2,5-diaza- | C | 4.10 | 432 | 0.41 |
| | bicyclo[2.2.1]heptane-2-carboxylic acid (4- | | | | |
| | phenoxy-phenyl)-amide | | | | |
| 52 | 5-[1-(4-Fluoro-phenyl)-ethyl]-2,5-diaza- | C | 4.17 | 432 | 0.90 |
| | bicyclo[2.2.1]heptane-2-carboxylic acid (3- | | | | |
| | phenoxy-phenyl)-amide | | | | |
| 53 | 5-[1-(4-Fluoro-phenyl)-ethyl]-2,5-diaza- | C | 4.02 | 408 | 2.50 |
| | bicyclo[2.2.1]heptane-2-carboxylic acid (4- | | | | |
| | trifluoromethyl-phenyl)-amide | | | | |
| 54 | 5-[1-(2-Trifluoromethyl-phenyl)-ethyl]-2,5- | C | 4.37 | 458 | 1.75 |
| | diaza-bicyclo[2.2.1]heptane-2-carboxylic acid | | | | |
| | (4-trifluoromethyl-phenyl)-amide | | | | |
| 55 | 5-[1-(2-Trifluoromethyl-phenyl)-ethyl]-2,5- | C | 4.36 | 458 | 4.24 |
| | diaza-bicyclo[2.2.1]heptane-2-carboxylic acid | | | | |
| | (3-trifluoromethyl-phenyl)-amide | | | | |
| 56 | 5-[1-(2-Trifluoromethyl-phenyl)-ethyl]-2,5- | C | 4.45 | 458 | 7.91 |
| | diaza-bicyclo[2.2.1]heptane-2-carboxylic acid | | | | |
| | (4-trifluoromethyl-phenyl)-amide | | | | |
| 57 | 5-[1-(2-Trifluoromethyl-phenyl)-ethyl]-2,5- | C | 4.45 | 458 | 5.13 |
| | diaza-bicyclo[2.2.1]heptane-2-carboxylic acid | | | | |
| | (3-trifluoromethyl-phenyl)-amide | | | | |
| 58 | 5-(1-Methyl-2-o-tolyl-ethyl)-2,5-diaza- | C | 4.09 | 442 | 0.23 |
| | bicyclo[2.2.1]heptane-2-carboxylic acid (4- | | | | |
| | phenoxy-phenyl)-amide | | | | |
| 59 | 5-(1-Methyl-2-o-tolyl-ethyl)-2,5-diaza- | C | 4.02 | 418 | 0.75 |
| | bicyclo[2.2.1]heptane-2-carboxylic acid (4- | | | | |
| | trifluoromethyl-phenyl)-amide | | | | |
| 60 | 5-[1-(2-Trifluoromethyl-phenyl)-ethyl]-2,5- | C | 4.44 | 482 | 1.48 |
| | diaza-bicyclo[2.2.1]heptane-2-carboxylic acid | | | | |
| | (4-phenoxy-phenyl)-amide | | | | |
| 61 | 5-[1-(2-Trifluoromethyl-phenyl)-ethyl]-2,5- | C | 4.52 | 482 | 4.64 |
| | diaza-bicyclo[2.2.1]heptane-2-carboxylic acid | | | | |
| | (3-phenoxy-phenyl)-amide | | | | |
| 62 | 5-[1-(2-Trifluoromethyl-phenyl)-ethyl]-2,5- | C | 4.55 | 482 | 17.23 |
| | diaza-bicyclo[2.2.1]heptane-2-carboxylic acid | | | | |
| | (4-phenoxy-phenyl)-amide | | | | |
| 63 | 5-[1-(2-Trifluoromethyl-phenyl)-ethyl]-2,5- | C | 4.55 | 482 | 6.10 |
| | diaza-bicyclo[2.2.1]heptane-2-carboxylic acid | | | | |
| | (3-phenoxy-phenyl)-amide | | | | |
| 64 | 5-[1-(4-Fluoro-2-methyl-phenyl)-ethyl]-2,5- | C | 4.30 | 446 | 0.27 |
| | diaza-bicyclo[2.2.1]heptane-2-carboxylic acid | | | | |
| | (4-phenoxy-phenyl)-amide | | | | |
| 65 | 5-[1-(4-Fluoro-2-methyl-phenyl)-ethyl]-2,5- | C | 4.24 | 422 | 0.60 |
| | diaza-bicyclo[2.2.1]heptane-2-carboxylic acid | | | | |
| | (4-trifluoromethyl-phenyl)-amide | | | | |
| 66 | 5-(1-o-Tolyl-ethyl)-2,5-diaza- | C | 4.07 | 440 | 0.72 |
| | bicyclo[2.2.1]heptane-2-carboxylic acid (4- | | | | |
| | benzoyl-phenyl)-amide | | | | |
| 67 | 5-(1-Methyl-2-o-tolyl-ethyl)-2,5-diaza- | C | 4.01 | 418 | 1.22 |
| | bicyclo[2.2.1]heptane-2-carboxylic acid (3- | | | | |
| | trifluoromethyl-phenyl)-amide | | | | |
| 68 | 5-(1-o-Tolyl-ethyl)-2,5-diaza- | Note² | 3.67 | 442 | 6.15 |
| | bicyclo[2.2.1]heptane-2-carboxylic acid [4- | | | | |
| | (hydroxy-phenyl-methyl)-phenyl]-amide | | | | |
| 69 | 5-(1-o-Tolyl-ethyl)-2,5-diaza- | C | 3.74 | 382 | 17.12 |
| | bicyclo[2.2.1]heptane-2-carboxylic acid [1-(4- | | | | |
| | fluoro-phenyl)-ethyl]-amide | | | | |
| 70 | 5-(1-Methyl-2-o-tolyl-ethyl)-2,5-diaza- | C | 3.65 | 396 | 5.08 |
| | bicyclo[2.2.1]heptane-2-carboxylic acid [1-(4- | | | | |
| | fluoro-phenyl)-ethyl]-amide | | | | |
| 71 | 5-[1-(4-Fluoro-2-methyl-phenyl)-ethyl]-2,5- | C | 4.14 | 458 | 2.36 |
| | diaza-bicyclo[2.2.1]heptane-2-carboxylic acid | | | | |
| | (4-benzoyl-phenyl)-amide | | | | |
| 72 | 5-[1-(4-Fluoro-2-methyl-phenyl)-ethyl]-2,5- | C | 4.13 | 458 | 2.26 |
| | diaza-bicyclo[2.2.1]heptane-2-carboxylic acid | | | | |
| | (4-benzoyl-phenyl)-amide | | | | |
| 73 | 5-[1-(2-Chloro-phenyl)-ethyl]-2,5-diaza- | B | 4.30 | 466 | 1.13 |
| | bicyclo[2.2.1]heptane-2-carboxylic acid [4-(4- | | | | |
| | fluoro-phenoxy)-phenyl]-amide | | | | |
| 74 | 5-(1-o-Tolyl-ethyl)-2,5-diaza- | B | 4.21 | 446 | 0.11 |
| | bicyclo[2.2.1]heptane-2-carboxylic acid [4-(4- | | | | |
| | fluoro-phenoxy)-phenyl]-amide | | | | |
| 75 | 5-[1-(4-Fluoro-2-methyl-phenyl)-ethyl]-2,5- | B | 4.30 | 464 | 0.57 |
| | diaza-bicyclo[2.2.1]heptane-2-carboxylic acid | | | | |
| | [4-(4-fluoro-phenoxy)-phenyl]-amide | | | | |
| 76 | 5-[1-(2-Chloro-phenyl)-ethyl]-2,5-diaza- | B | 4.30 | 464 | 0.27 |
| | bicyclo[2.2.1]heptane-2-carboxylic acid [4-(4- | | | | |
| | fluoro-phenoxy)-phenyl]-amide | | | | |
| 77 | 5-(1,2,3,4-Tetrahydro-naphthalen-2-yl)-2,5- | A | 3.94 | 416 | 0.59 |
| | diaza-bicyclo[2.2.1]heptane-2-carboxylic acid | | | | |
| | (4-trifluoromethyl-phenyl)-amide | | | | |
| 78 | 5-(1-o-Tolyl-ethyl)-2,5-diaza- | Salt³ | 4.20 | 446 | 0.23 |
| | bicyclo[2.2.1]heptane-2-carboxylic acid [4-(4- | | | | |
| | fluoro-phenoxy)-phenyl]-amide fumarate salt | | | | |
| 79 | 5-[2-(2-Fluoro-phenyl)-1-methyl-ethyl]-2,5- | A | 3.87 | 422 | 0.78 |
| | diaza-bicyclo[2.2.1]heptane-2-carboxylic acid | | | | |
| | (4-trifluoromethyl-phenyl)-amide | | | | |
| 80 | 5-[2-(4-Fluoro-phenyl)-1-methyl-ethyl]-2,5- | A | 3.87 | 422 | 0.49 |
| | diaza-bicyclo[2.2.1]heptane-2-carboxylic acid | | | | |
| | (4-trifluoromethyl-phenyl)-amide | | | | |
| 81 | 5-(2-Cyano-1-methyl-2-phenyl-ethyl)-2,5-diaza- | A | 3.83/3.93 | 427 | 26.19 |
| | bicyclo[2.2.1]heptane-2-carboxylic acid (4- | | (2 isomers) | | |
| | trifluoromethyl-phenyl)-amide | | | | |
| 82 | 5-[2-(2-Chloro-phenyl)-1-methyl-ethyl]-2,5- | A | 4.00 | 439 | 0.44 |
| | diaza-bicyclo[2.2.1]heptane-2-carboxylic acid | | | | |
| | (4-trifluoromethyl-phenyl)-amide | | | | |
| 83 | 5-[2-(4-Chloro-phenyl)-1-methyl-ethyl]-2,5- | A | 4.09 | 438 | 0.68 |
| | diaza-bicyclo[2.2.1]heptane-2-carboxylic acid | | | | |
| | (4-trifluoromethyl-phenyl)-amide | | | | |
| 84 | 5-[2-(2,4-Dichloro-phenyl)-1-methyl-ethyl]-2,5- | A | 4.23 | 472 | 1.06 |
| | diaza-bicyclo[2.2.1]heptane-2-carboxylic acid | | | | |
| | (4-trifluoromethyl-phenyl)-amide | | | | |
| 85 | 5-[2-(2-Chloro-6-fluoro-phenyl)-1-methyl- | A | 3.99/4.07 | 456 | 0.63 |
| | ethyl]-2,5-diaza-bicyclo[2.2.1]heptane-2- | | 2 isomers | | |
| | carboxylic acid (4-trifluoromethyl-phenyl)- | | | | |
| | amide | | | | |
| 86 | 5-[2-(2-Methoxy-phenyl)-1-methyl-ethyl]-2,5- | A | 3.89 | 434 | 0.67 |
| | diaza-bicyclo[2.2.1]heptane-2-carboxylic acid | | | | |
| | (4-trifluoromethyl-phenyl)-amide | | | | |
| 87 | 5-(1-o-Tolyl-ethyl)-2,5-diaza- | Salt³ | 4.19 | 446 | 0.60 |
| | bicyclo[2.2.1]heptane-2-carboxylic acid [4-(4- | | | | |
| | fluoro-phenoxy)-phenyl]-amide | | | | |
| | methanesulfonate salt | | | | |
| 88 | 7-Benzyl-2,7-diaza-spiro[4.4]nonane-2- | E | 4.08 | 428 | 0.39 |
| | carboxylic acid (4-phenoxy-phenyl)-amide | | | | |
| 89 | 5-Benzyl-hexahydro-pyrrolo[3,4-c]pyrrole-2- | E | 4.09 | 414 | 0.40 |
| | carboxylic acid (4-phenoxy-phenyl)-amide | | | | |
| 90 | 5-[1-(4-Cyano-phenyl)-ethyl]-2,5-diaza- | B | 3.93 | 455 | 1.40 |
| | bicyclo[2.2.1]heptane-2-carboxylic acid [4-(4- | | | | |
| | fluoro-phenoxy)-phenyl]-amide | | | | |
| 91 | 5-[1-(4-Cyano-phenyl)-ethyl]-2,5-diaza- | B | 3.91 | 455 | 1.05 |
| | bicyclo[2.2.1]heptane-2-carboxylic acid [4-(4- | | | | |
| | fluoro-phenoxy)-phenyl]-amide | | | | |
| 92 | 5-(5-Fluoro-indan-1-yl)-2,5-diaza- | B | 4.01 | 462 | 0.36 |
| | bicyclo[2.2.1]heptane-2-carboxylic acid [4-(4- | | | | |
| | fluoro-phenoxy)-phenyl]-amide | | | | |
| 93 | 5-(5-Fluoro-indan-1-yl)-2,5-diaza- | B | 4.13 | 462 | 0.31 |
| | bicyclo[2.2.1]heptane-2-carboxylic acid [4-(4- | | | | |
| | fluoro-phenoxy)-phenyl]-amide | | | | |
| 94 | 5-(5-Fluoro-indan-1-yl)-2,5-diaza- | C | 4.03 | 420 | 1.89 |
| | bicyclo[2.2.1]heptane-2-carboxylic acid (4- | | | | |
| | trifluoromethyl-phenyl)-amide | | | | |
| 95 | 5-(5-Fluoro-indan-1-yl)-2,5-diaza- | C | 3.92 | 420 | 1.06 |
| | bicyclo[2.2.1]heptane-2-carboxylic acid (4- | | | | |
| | trifluoromethyl-phenyl)-amide | | | | |
| 96 | 5-[1-(2-Chloro-phenyl)-ethyl]-2,5-diaza- | Salt³ | 4.35 | 467 | 0.37 |
| | bicyclo[2.2.1]heptane-2-carboxylic acid [4-(4- | | | | |
| | fluoro-phenoxy)-phenyl]-amide | | | | |
| | methanesulfonate salt | | | | |
| 97 | 5-(1,2,3,4-Tetrahydro-naphthalen-2-yl)-2,5- | B | 4.06 | 458 | 0.31 |
| | diaza-bicyclo[2.2.1]heptane-2-carboxylic acid | | | | |
| | [4-(4-fluoro-phenoxy)-phenyl]-amide | | | | |
| 98 | 5-Benzyl-hexahydro-pyrrolo[3,4-c]pyrrole-2- | F(i) | 4.09 | 432 | 0.17 |
| | carboxylic acid [4-(4-fluoro-phenoxy)-phenyl]- | | | | |
| | amide | | | | |
| 99 | 5-Indan-2-yl-2,5-diaza-bicyclo[2.2.1]heptane-2- | C | 3.83 | 402 | 2.38 |
| | carboxylic acid (4-trifluoromethyl-phenyl)- | | | | |
| | amide | | | | |
| 100 | 5-(6-Fluoro-1,2,3,4-tetrahydro-naphthalen-2-yl)- | C | 3.96 | 434 | 0.64 |
| | 2,5-diaza-bicyclo[2.2.1]heptane-2-carboxylic | | | | |
| | acid (4-trifluoromethyl-phenyl)-amide | | | | |
| 101 | 5-(1,2,3,4-Tetrahydro-naphthalen-2-yl)-2,5- | Salt³ | 4.20 | | 0.34 |
| | diaza-bicyclo[2.2.1]heptane-2-carboxylic acid | | | | |
| | [4-(4-fluoro-phenoxy)-phenyl]-amide | | | | |
| | methanesulfonate salt | | | | |
| 102 | 5-(6-Fluoro-1,2,3,4-tetrahydro-naphthalen-2-yl)- | B | 4.06 | 476 | 0.71 |
| | 2,5-diaza-bicyclo[2.2.1]heptane-2-carboxylic | | | | |
| | acid [4-(4-fluoro-phenoxy)-phenyl]-amide | | | | |
| 103 | 7-Benzyl-2,7-diaza-spiro[4.4]nonane-2- | F(i) | 4.14 | 446 | 0.39 |
| | carboxylic acid [4-(4-fluoro-phenoxy)-phenyl]- | | | | |
| | amide | | | | |
| 104 | 7-Indan-2-yl-2,7-diaza-spiro[4.4]nonane-2- | F | 4.18 | 472 | 0.43 |
| | carboxylic acid [4-(4-fluoro-phenoxy)-phenyl]- | | | | |
| | amide | | | | |
| 105 | 5-Indan-2-yl-2,5-diaza-bicyclo[2.2.1]heptane-2- | C | 3.89 | 426 | 0.20 |
| | carboxylic acid (4-phenoxy-phenyl)-amide | | | | |
| 106 | 5-Indan-2-yl-2,5-diaza-bicyclo[2.2.1]heptane-2- | B | 3.92 | 444 | 0.17 |
| | carboxylic acid [4-(4-fluoro-phenoxy)-phenyl]- | | | | |
| | amide | | | | |
| 107 | 5-[1-(2-Chloro-phenyl)-ethyl]-hexahydro- | F | 4.48 | 480 | 1.13 |
| | pyrrolo[3,4-c]pyrrole-2-carboxylic acid [4-(4- | | | | |
| | fluoro-phenoxy)-phenyl]-amide | | | | |
| 108 | 5-[1-(2-Chloro-phenyl)-ethyl]-2,5-diaza- | B | 4.34 | 466 | 0.60 |
| | bicyclo[2.2.1]heptane-2-carboxylic acid [4-(3- | | | | |
| | fluoro-phenoxy)-phenyl]-amide | | | | |
| 109 | 5-[1-(2-Chloro-phenyl)-ethyl]-2,5-diaza- | B | 4.24 | 466 | 0.68 |
| | bicyclo[2.2.1]heptane-2-carboxylic acid [4-(2- | | | | |
| | fluoro-phenoxy)-phenyl]-amide | | | | |
| 110 | 5-[1-(2-Chloro-phenyl)-ethyl]-2,5-diaza- | B | 4.34 | 484 | 0.21 |
| | bicyclo[2.2.1]heptane-2-carboxylic acid [3- | | | | |
| | fluoro-4-(4-fluoro-phenoxy)-phenyl]-amide | | | | |
| 111 | 7-[1-(2-Chloro-phenyl)-ethyl]-2,7-diaza- | F | 4.55 | 494 | 0.38 |
| | spiro[4.4]nonane-2-carboxylic acid [4-(4-fluoro- | | | | |
| | phenoxy)-phenyl]-amide | | | | |
| 112 | 5-Indan-2-yl-hexahydro-pyrrolo[3,4-c]pyrrole- | F | 4.12 | 458 | 0.30 |
| | 2-carboxylic acid [4-(4-fluoro-phenoxy)- | | | | |
| | phenyl]-amide | | | | |
| 113 | 5-Indan-2-yl-2,5-diaza-bicyclo[2.2.1]heptane-2- | B | 3.88 | 444 | 0.54 |
| | carboxylic acid [4-(2-fluoro-phenoxy)-phenyl]- | | | | |
| | amide | | | | |
| 114 | 5-Indan-2-yl-2,5-diaza-bicyclo[2.2.1]heptane-2- | B | 3.98 | 444 | 0.08 |
| | carboxylic acid [4-(3-fluoro-phenoxy)-phenyl]- | | | | |
| | amide | | | | |
| 115 | 5-[1-(2-Chloro-phenyl)-ethyl]-2,5-diaza- | D | 4.51 | 480 | 1.98 |
| | bicyclo[2.2.1]heptane-2-carboxylic acid [4-(4- | | | | |
| | fluoro-phenoxy)-phenyl]-methyl-amide | | | | |
| 116 | 5-[1-(2,4-Dimethyl-phenyl)-ethyl]-2,5-diaza- | B | 4.41 | 460 | 0.29 |
| | bicyclo[2.2.1]heptane-2-carboxylic acid [4-(4- | | | | |
| | fluoro-phenoxy)-phenyl]-amide | | | | |
| 117 | 5-[1-(2,4-Dimethyl-phenyl)-ethyl]-2,5-diaza- | B | 4.38 | 460 | 0.42 |
| | bicyclo[2.2.1]heptane-2-carboxylic acid [4-(4- | | | | |
| | fluoro-phenoxy)-phenyl]-amide | | | | |
| 118 | 5-[1-(2,4-Dichloro-phenyl)-ethyl]-2,5-diaza- | B | 4.64 | 500 | 17.58 |
| | bicyclo[2.2.1]heptane-2-carboxylic acid [4-(4- | | | | |
| | fluoro-phenoxy)-phenyl]-amide | | | | |
| 119 | 5-[1-(2,4-Dichloro-phenyl)-ethyl]-2,5-diaza- | B | 4.64 | 500 | 20.95 |
| | bicyclo[2.2.1]heptane-2-carboxylic acid [4-(4- | | | | |
| | fluoro-phenoxy)-phenyl]-amide | | | | |
| 120 | 5-[1-(2,4-Dichloro-phenyl)-ethyl]-2,5-diaza- | C | 4.55 | 458 | 24.47 |
| | bicyclo[2.2.1]heptane-2-carboxylic acid (4- | | | | |
| | trifluoromethyl-phenyl)-amide | | | | |
| 121 | 5-[1-(2,4-Dichloro-phenyl)-ethyl]-2,5-diaza- | B | 4.84 | 518 | 22.37 |
| | bicyclo[2.2.1]heptane-2-carboxylic acid [4-(4- | | | | |
| | chloro-phenoxy)-phenyl]-amide | | | | |
| 122 | 5-[1-(2,4-Dichloro-phenyl)-ethyl]-2,5-diaza- | B | 4.85 | 518 | 19.15 |
| | bicyclo[2.2.1]heptane-2-carboxylic acid [4-(4- | | | | |
| | chloro-phenoxy)-phenyl]-amide | | | | |
| 123 | 5-[1-(2,4-Dichloro-phenyl)-ethyl]-2,5-diaza- | B | 4.78 | 496 | 20.77 |
| | bicyclo[2.2.1]heptane-2-carboxylic acid (4-p- | | | | |
| | tolyloxy-phenyl)-amide | | | | |
| 124 | 5-(5-Fluoro-indan-2-yl)-2,5-diaza- | C | 4.01 | 442 | 0.64 |
| | bicyclo[2.2.1]heptane-2-carboxylic acid (4- | | | | |
| | benzyl-phenyl)-amide | | | | |
| 125 | 5-(5-Fluoro-indan-2-yl)-2,5-diaza- | C | 3.92 | 444 | 0.78 |
| | bicyclo[2.2.1]heptane-2-carboxylic acid (4- | | | | |
| | phenoxy-phenyl)-amide | | | | |
| 126 | 5-[1-(2,4-Dichloro-phenyl)-ethyl]-2,5-diaza- | C | 4.56 | 458 | 22.89 |
| | bicyclo[2.2.1]heptane-2-carboxylic acid (4- | | | | |
| | trifluoromethyl-phenyl)-amide | | | | |
| 127 | 5-[1-(2,4-Dichloro-phenyl)-ethyl]-2,5-diaza- | B | 4.78 | 496 | 22.07 |
| | bicyclo[2.2.1]heptane-2-carboxylic acid (4-p- | | | | |
| | tolyloxy-phenyl)-amide | | | | |
| 128 | 5-[1-(2-Chloro-phenyl)-ethyl]-2,5-diaza- | B | 4.52 | 482 | 1.42 |
| | bicyclo[2.2.1]heptane-2-carboxylic acid [4-(4- | | | | |
| | chloro-phenoxy)-phenyl]-amide | | | | |
| 129 | 5-[1-(2-Chloro-phenyl)-ethyl]-2,5-diaza- | B | 4.52 | 482 | 3.80 |
| | bicyclo[2.2.1]heptane-2-carboxylic acid [4-(4- | | | | |
| | chloro-phenoxy)-phenyl]-amide | | | | |
| 130 | 5-(5-Fluoro-indan-2-yl)-2,5-diaza- | C | 3.86 | 420 | 3.61 |
| | bicyclo[2.2.1]heptane-2-carboxylic acid (4- | | | | |
| | trifluoromethyl-phenyl)-amide | | | | |
| 131 | 5-(5-Fluoro-indan-2-yl)-2,5-diaza- | B | 3.96 | 462 | 0.55 |
| | bicyclo[2.2.1]heptane-2-carboxylic acid [4-(4- | | | | |
| | fluoro-phenoxy)-phenyl]-amide | | | | |
| 132 | 5-[1-(2-Chloro-4-fluoro-phenyl)-ethyl]-2,5- | B | 4.39 | 484 | 1.14 |
| | diaza-bicyclo[2.2.1]heptane-2-carboxylic acid | | | | |
| | [4-(4-fluoro-phenoxy)-phenyl]-amide | | | | |
| 133 | 5-[1-(2-Chloro-4-fluoro-phenyl)-ethyl]-2,5- | B | 4.37 | 484 | 1.19 |
| | diaza-bicyclo[2.2.1]heptane-2-carboxylic acid | | | | |
| | [4-(4-fluoro-phenoxy)-phenyl]-amide | | | | |
| 134 | 1-[5-(5-Fluoro-indan-2-yl)-2,5-diaza- | G | 3.93 | 443 | 1.60 |
| | bicyclo[2.2.1]hept-2-yl]-2-(4-phenoxy-phenyl)- | | | | |
| | ethanone | | | | |
| 135 | 1-{5-[1-(2-Chloro-phenyl)-ethyl]-2,5-diaza- | G | 4.36 | 465 | 0.94 |
| | bicyclo[2.2.1]hept-2-yl}-2-[4-(4-fluoro- | | | | |
| | phenoxy)-phenyl]-ethanone | | | | |
| 136 | 1-{5-[1-(2-Chloro-phenyl)-ethyl]-2,5-diaza- | G | 4.37 | 465 | 2.41 |
| | bicyclo[2.2.1]hept-2-yl}-2-[4-(4-fluoro- | | | | |
| | phenoxy)-phenyl]-ethanone | | | | |
| 137 | [5-(1,2,3,4-Tetrahydro-naphthalen-2-yl)-2,5- | G | 3.85 | 401 | 23.16 |
| | diaza-bicyclo[2.2.1]hept-2-yl]-(4- | | | | |
| | trifluoromethyl-phenyl)-methanone | | | | |
| 138 | [5-(7-Fluoro-1,2,3,4-tetrahydro-naphthalen-2- | G | 3.89 | 419 | 13.32 |
| | yl)-2,5-diaza-bicyclo[2.2.1]hept-2-yl]-(4- | | | | |
| | trifluoromethyl-phenyl)-methanone | | | | |
| 139 | 5-[1-(2-Chloro-phenyl)-ethyl]-2,5-diaza- | B | 4.42 | 462 | 1.16 |
| | bicyclo[2.2.1]heptane-2-carboxylic acid (4-p- | | | | |
| | tolyloxy-phenyl)-amide | | | | |
| 140 | 5-[1-(2-Chloro-phenyl)-ethyl]-2,5-diaza- | B | 4.43 | 462 | 1.23 |
| | bicyclo[2.2.1]heptane-2-carboxylic acid (4-p- | | | | |
| | tolyloxy-phenyl)-amide | | | | |
| 141 | 5-(7-Chloro-indan-1-yl)-2,5-diaza- | C | 4.40 | 460 | 0.20 |
| | bicyclo[2.2.1]heptane-2-carboxylic acid (4- | | | | |
| | phenoxy-phenyl)-amide | | | | |
| 142 | 2-[4-(4-Fluoro-phenoxy)-phenyl]-1-[5-(1,2,3,4- | G | 4.03 | 457 | 0.28 |
| | tetrahydro-naphthalen-2-yl)-2,5-diaza- | | | | |
| | bicyclo[2.2.1]hept-2-yl]-ethanone | | | | |
| 143 | 5-[1-(2-Chloro-phenyl)-ethyl]-2,5-diaza- | D | 4.60 | 480 | 3.21 |
| | bicyclo[2.2.1]heptane-2-carboxylic acid [4-(4- | | | | |
| | fluoro-phenoxy)-phenyl]-methyl-amide | | | | |
| 144 | 2-[4-(4-Fluoro-phenoxy)-phenyl]-1-[5-(6-fluoro- | G | 4.06 | 475 | 1.60 |
| | 1,2,3,4-tetrahydro-naphthalen-2-yl)-2,5-diaza- | | | | |
| | bicyclo[2.2.1]hept-2-yl]-ethanone | | | | |
| 145 | 5-(5-Fluoro-indan-2-yl)-2,5-diaza- | B | 3.86 | 458 | 1.44 |
| | bicyclo[2.2.1]heptane-2-carboxylic acid 4- | | | | |
| | phenoxy-benzylamide | | | | |
| 146 | 5-(5-Fluoro-indan-2-yl)-2,5-diaza- | B | 3.87 | 476 | 1.28 |
| | bicyclo[2.2.1]heptane-2-carboxylic acid 4-(4- | | | | |
| | fluoro-phenoxy)-benzylamide | | | | |
| 147 | 1-[5-(1,2,3,4-Tetrahydro-naphthalen-2-yl)-2,5- | G | 3.82 | 415 | 3.10 |
| | diaza-bicyclo[2.2.1]hept-2-yl]-2-(4- | | | | |
| | trifluoromethyl-phenyl)-ethanone | | | | |
| 148 | 1-[5-(7-Fluoro-1,2,3,4-tetrahydro-naphthalen-2- | G | 3.84 | 433 | 2.39 |
| | yl)-2,5-diaza-bicyclo[2.2.1]hept-2-yl]-2-(4- | | | | |
| | trifluoromethyl-phenyl)-ethanone | | | | |
| 149 | {5-[1-(2-Chloro-phenyl)-ethyl]-2,5-diaza- | G | 4.15 | 380 | 5.84 |
| | bicyclo[2.2.1]hept-2-yl}-(1H-indol-2-yl)- | | | | |
| | methanone | | | | |
| 150 | (5-Chloro-1H-indol-2-yl)-{5-[1-(2-chloro- | G | 4.36 | 414 | 18.18 |
| | phenyl)-ethyl]-2,5-diaza-bicyclo[2.2.1]hept-2- | | | | |
| | yl}-methanone | | | | |
| 151 | {5-[1-(2-Chloro-phenyl)-ethyl]-2,5-diaza- | G | 4.39 | 451 | 1.11 |
| | bicyclo[2.2.1]hept-2-yl}-[4-(4-fluoro-phenoxy)- | | | | |
| | phenyl]-methanone | | | | |
| 152 | {5-[1-(2-Chloro-phenyl)-ethyl]-2,5-diaza- | G | 4.34 | 451 | 2.52 |
| | bicyclo[2.2.1]hept-2-yl}-[4-(4-fluoro-phenoxy)- | | | | |
| | phenyl]-methanone | | | | |
| 153 | 1-{5-[1-(2-Bromo-phenyl)-ethyl]-2,5-diaza- | G | 4.43 | 510 | 3.33 |
| | bicyclo[2.2.1]hept-2-yl}-2-[4-(4-fluoro- | | | | |
| | phenoxy)-phenyl]-ethanone | | | | |
| 154 | 1-{5-[1-(2-Bromo-phenyl)-ethyl]-2,5-diaza- | G | 4.45 | 510 | 4.31 |
| | bicyclo[2.2.1]hept-2-yl}-2-[4-(4-fluoro- | | | | |
| | phenoxy)-phenyl]-ethanone | | | | |
| 155 | 1-{5-[1-(2,4-Dichloro-phenyl)-ethyl]-2,5-diaza- | G | 4.69 | 500 | 18.24 |
| | bicyclo[2.2.1]hept-2-yl}-2-[4-(4-fluoro- | | | | |
| | phenoxy)-phenyl]-ethanone | | | | |
| 156 | 1-{5-[1-(2,4-Dichloro-phenyl)-ethyl]-2,5-diaza- | G | 4.74 | 500 | |
| | bicyclo[2.2.1]hept-2-yl}-2-[4-(4-fluoro- | | | | |
| | phenoxy)-phenyl]-ethanone | | | | |
| 157 | 1-{5-[1-(2,5-Dichloro-phenyl)-ethyl]-2,5-diaza- | G | 4.66 | 500 | 30.00 |
| | bicyclo[2.2.1]hept-2-yl}-2-[4-(4-fluoro- | | | | |
| | phenoxy)-phenyl]-ethanone | | | | |
| 158 | 1-{5-[1-(2,5-Dichloro-phenyl)-ethyl]-2,5-diaza- | G | 4.68 | 500 | 30.00 |
| | bicyclo[2.2.1]hept-2-yl}-2-[4-(4-fluoro- | | | | |
| | phenoxy)-phenyl]-ethanone | | | | |
| 159 | {5-[1-(2-Bromo-phenyl)-ethyl]-2,5-diaza- | G | 4.48 | 478 | 14.76 |
| | bicyclo[2.2.1]hept-2-yl}-(4-phenoxy-phenyl)- | | | | |
| | methanone | | | | |
| 160 | {5-[1-(2-Bromo-phenyl)-ethyl]-2,5-diaza- | G | 4.41 | 478 | |
| | bicyclo[2.2.1]hept-2-yl}-(4-phenoxy-phenyl)- | | | | |
| | methanone | | | | |
| 161 | 1-{5-[1-(2-Chloro-4-fluoro-phenyl)-ethyl]-2,5- | G | 4.42 | 483 | 4.75 |
| | diaza-bicyclo[2.2.1]hept-2-yl}-2-[4-(4-fluoro- | | | | |
| | phenoxy)-phenyl]-ethanone | | | | |
| 162 | 1-{5-[1-(2-Chloro-4-fluoro-phenyl)-ethyl]-2,5- | G | 4.63 | 483 | 18.29 |
| | diaza-bicyclo[2.2.1]hept-2-yl}-2-[4-(4-fluoro- | | | | |
| | phenoxy)-phenyl]-ethanone | | | | |
| 163 | {5-[1-(2-Chloro-4-fluoro-phenyl)-ethyl]-2,5- | G | 4.51 | 469 | 17.69 |
| | diaza-bicyclo[2.2.1]hept-2-yl}-[4-(4-fluoro- | | | | |
| | phenoxy)-phenyl]-methanone | | | | |
| 164 | {5-[1-(2-Chloro-4-fluoro-phenyl)-ethyl]-2,5- | G | 4.43 | 469 | 13.50 |
| | diaza-bicyclo[2.2.1]hept-2-yl}-[4-(4-fluoro- | | | | |
| | phenoxy)-phenyl]-methanone | | | | |
| 165 | 1-{5-[1-(2-Chloro-6-fluoro-phenyl)-ethyl]-2,5- | G | 4.29 | 483 | 1.23 |
| | diaza-bicyclo[2.2.1]hept-2-yl}-2-[4-(4-fluoro- | | | | |
| | phenoxy)-phenyl]-ethanone | | | | |
| 166 | 1-{5-[1-(2-Chloro-6-fluoro-phenyl)-ethyl]-2,5- | G | 4.26 | 483 | 3.91 |
| | diaza-bicyclo[2.2.1]hept-2-yl}-2-[4-(4-fluoro- | | | | |
| | phenoxy)-phenyl]-ethanone | | | | |
| 167 | {5-[1-(2-Chloro-6-fluoro-phenyl)-ethyl]-2,5- | G | 4.33 | 469 | 2.22 |
| | diaza-bicyclo[2.2.1]hept-2-yl}-[4-(4-fluoro- | | | | |
| | phenoxy)-phenyl]-methanone | | | | |
| 168 | {5-[1-(2-Chloro-6-fluoro-phenyl)-ethyl]-2,5- | G | 4.22 | 469 | 1.70 |
| | diaza-bicyclo[2.2.1]hept-2-yl}-[4-(4-fluoro- | | | | |
| | phenoxy)-phenyl]-methanone | | | | |
| 169 | {5-[1-(2-Chloro-phenyl)-ethyl]-2,5-diaza- | G | 4.34 | 451 | 0.84 |
| | bicyclo[2.2.1]hept-2-yl}-[4-(3-fluoro-phenoxy)- | | | | |
| | phenyl]-methanone | | | | |
| 170 | 1-{5-[1-(2-Chloro-phenyl)-ethyl]-2,5-diaza- | G | 4.34 | 465 | 1.57 |
| | bicyclo[2.2.1]hept-2-yl}-2-[4-(3-fluoro- | | | | |
| | phenoxy)-phenyl]-ethanone | | | | |
| 171 | 2-[4-(3-Fluoro-phenoxy)-phenyl]-1-[5-(1-o- | G | 4.26 | 445 | 0.67 |
| | tolyl-ethyl)-2,5-diaza-bicyclo[2.2.1]hept-2-yl]- | | | | |
| | ethanone | | | | |
| 172 | 2-[4-(3-Fluoro-phenoxy)-phenyl]-1-[5-(1-o- | G | 4.30 | 445 | 0.71 |
| | tolyl-ethyl)-2,5-diaza-bicyclo[2.2.1]hept-2-yl]- | | | | |
| | ethanone | | | | |
| 173 | 3-(4-{5-[1-(2-Chloro-phenyl)-ethyl]-2,5-diaza- | G | 4.09 | 458 | 1.93 |
| | bicyclo[2.2.1]heptane-2-carbonyl}-phenoxy)- | | | | |
| | benzonitrile | | | | |
| 174 | 3-(4-{5-[1-(2-Chloro-phenyl)-ethyl]-2,5-diaza- | G | 4.26 | 458 | 1.40 |
| | bicyclo[2.2.1]heptane-2-carbonyl}-phenoxy)- | | | | |
| | benzonitrile | | | | |
| 175 | {5-[1-(2-Chloro-phenyl)-ethyl]-2,5-diaza- | G | 4.52 | 451 | 0.64 |
| | bicyclo[2.2.1]hept-2-yl}-[4-(3-fluoro-phenoxy)- | | | | |
| | phenyl]-methanone | | | | |
| 176 | {5-[1-(2-Chloro-phenyl)-ethyl]-2,5-diaza- | G | 4.45 | 469 | 0.43 |
| | bicyclo[2.2.1]hept-2-yl}-[3-fluoro-4-(4-fluoro- | | | | |
| | phenoxy)-phenyl]-methanone | | | | |
| 177 | {5-[1-(2-Chloro-phenyl)-ethyl]-2,5-diaza- | G | 4.39 | 469 | 0.74 |
| | bicyclo[2.2.1]hept-2-yl}-[3-fluoro-4-(4-fluoro- | | | | |
| | phenoxy)-phenyl]-methanone | | | | |
| 178 | {5-[1-(2-Chloro-phenyl)-ethyl]-2,5-diaza- | Salt³ | | | 0.61 |
| | bicyclo[2.2.1]hept-2-yl}-[3-fluoro-4-(4-fluoro- | | | | |
| | phenoxy)-phenyl]-methanone methanesulfonate | | | | |
| | salt | | | | |
| 179 | {5-[1-(2-Chloro-phenyl)-ethyl]-2,5-diaza- | Salt³ | | | 1.34 |
| | bicyclo[2.2.1]hept-2-yl}-[3-fluoro-4-(4-fluoro- | | | | |
| | phenoxy)-phenyl]-methanone methanesulfonate | | | | |
| | salt | | | | |
| 180 | 3-{4-[5-(1-Pyridin-2-yl-ethyl)-2,5-diaza- | G | 3.26 | 425 | 4.65 |
| | bicyclo[2.2.1]heptane-2-carbonyl]-phenoxy}- | | | | |
| | benzonitrile | | | | |
| 181 | [4-(4-Fluoro-phenoxy)-phenyl]-[5-(1-pyridin-2- | G | 3.47 | 418 | 9.93 |
| | yl-ethyl)-2,5-diaza-bicyclo[2.2.1]hept-2-yl]- | | | | |
| | methanone | | | | |
| 182 | 5-[1-(2-Chloro-phenyl)-ethyl]-2,5-diaza- | C³ | 3.96 | 467 | 0.27 |
| | bicyclo[2.2.1]heptane-2-carboxylic acid [6-(4- | | | | |
| | fluoro-phenoxy)-pyridin-3-yl]-amide | | | | |
| | methanesulfonate salt | | | | |

Notes:
¹Solvent: CH₃CN/H₂O/0.05% NH₃, 5-95% gradient H₂O-6 min. Column: Xterra 50 × 4.60 i.d., C18 reverse phase. Flow rate: 1.5 mL/min.
²Prepared by reduction of the ketone - Example 66
³Salts were typically prepared by evaporation of an equimolar solution of the parent compound and appropriate acid in DCM, followed by trituration with ether.

1. A compound of formula (I) for use in the treatment or prevention of a condition involving sodium ion flux through a sensory neurone specific channel of a sensory neurone, comprising 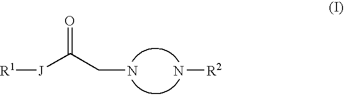
wherein: 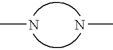
represents (A), (B) or (C) 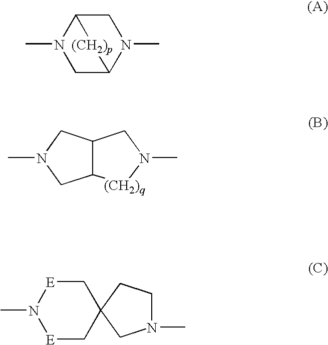
R¹ represents:
(a) -L-A or -L′-A′ wherein L represents a bond or a C₁-C₆ alkyl, C₂-C₆ alkenyl or C₂-C₆ alkynyl moiety, A represents a phenyl, 5- to 10-membered heteroaryl, C₃-C₆ carbocyclyl or 5- to 10-membered heterocyclyl group, L′ represents a C₁-C₆ alkyl, C₂-C₆ alkenyl or C₂-C₆ alkynyl moiety, and A′ represents -Het-A or —X-A wherein Het represents —O—, —S— or —NR′—, and X represents —CO—, —SO—, —SO₂—, —CO—O—, —CO—S—, —CONR′—, —O—CO—, —S—CO— or —NR′—CO—, wherein R′ represents hydrogen or C₁-C₆ alkyl;
(b) -L-A-A′ or -L-A-L-A wherein A′ is as defined above, each A is the same or different and is as defined above and each L is the same or different and is as defined above;
(c) -A-Z-A wherein Z is -Het-L′-, —X-L′-, -L′-Het- or -L′-X—, wherein Het, L′ and X are as defined above and each A is the same or different and is as defined above;
(d) -A-Het-Y or -A-X—Y wherein Y is -[L′-Het]ₙ-L′, -[L′-Het]_{n′}-A, -L′-B-L′, -L′-B-A or -A-L-A wherein n is from 1 to 4 and B is —X—, —NR′—CO—NR′—, —O—CO—NR′— or —NR′—CO—O—, and wherein X and L are as defined above, each A is the same or different and is as defined above, each L′ is the same or different and is as defined above, each R′ is the same or different and is as defined above and each Het is the same or different and is as defined above; or
(e) -L-CR(A)(A′) or -L-CR(A)(L-A) wherein R is hydrogen or C₁-C₄ alkyl, A′ is as defined above, each L is the same or different and is as defined above and each A is the same or different and is as defined above;
R² represents -L-A, -L′-A′, -L-A-A′ or -L-A-L-A wherein L′ and A′ are as defined above, each L is the same or different and is as defined above and each A is the same or different and is as defined above
J represents —NR³—, —O— or a direct bond wherein R³ represents hydrogen, C₁-C₆ alkyl, C₂-C₆ alkenyl or C₂-C₆ alkynyl;
p is an integer from 1 to 3;
q is 1 or 2; and
one of E and E′ is —CH₂— and the other is a direct bond;
wherein:
said phenyl, carbocyclyl, heterocyclyl and heteroaryl groups are optionally fused to a further cyclic moiety selected from phenyl, C₅-C₆ carbocyclyl, 5- to 6-membered heterocyclyl and 5- to 6-membered heteroaryl groups;
the phenyl, heteroaryl, carbocyclyl and heterocyclyl groups and moieties in the groups R¹ and R² are unsubstituted or substituted by one, two or three substituents which are the same or different and are selected from halogen, hydroxy, amino, thio, nitro, cyano, C₁-C₆ alkyl, C₂-C₆ alkenyl or -Het-L′, wherein Het and L′ are as defined above; and
the alkyl, alkenyl and alkynyl groups and moieties in R¹ to R³ are unsubstituted or substituted by one, two or three substituents which are the same or different and are selected from halogen, hydroxy, amino, thio and cyano substituents, or a pharmaceutically acceptable salt thereof.

2. A compound according to claim 1, wherein 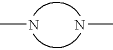
represents (A*), (B*) or (C*)

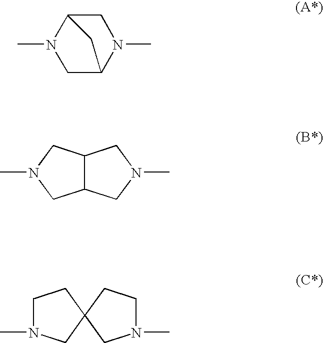

3. A compound according to claim 1, wherein X represents —CO—, —CO—O—, —CO—S— or —CONR′—.

4. A compound according to claim 1, wherein L represents a bond or a C₁-C₆ alkyl or C₂-C₆ alkenyl moiety.

5. A compound according to claim 1, wherein L′ represents a C₁-C₆ alkyl or C₂-C₆ alkenyl moiety.

6. A compound according to claim 1, wherein A represents a phenyl, 5- to 6-membered heteroaryl, C₃-C₆ carbocyclyl or 5- to 6-membered heterocyclyl group, said group being optionally fused to a phenyl, 5- to 6-membered heteroaryl, C₅-C₆ carbocyclyl or 5- to 6-membered heterocyclyl moiety.

7. A compound according to claim 1, wherein Z is -Het-L′- or —X-L′-.

8. A compound according to claim 1, wherein Y is -[L′-Het]_{n′}-L′, -L′-B-L′ or -A-L-A.

9. A compound according to claim 1, wherein R¹ represents: (a) -L-A; (b) -L-A-A′ or -A-L-A; (c) -A-Z-A; or (d) -A-Het-Y.

10. A compound according to claim 1, R³ represents hydrogen or C₁-C₄ alkyl.

11. A compound according to claim 1, wherein J is a direct bond or —NR³—.

12. A compound according to claim 1, wherein R² represents -L-A.

13. A compound according to claim 1, wherein 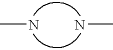
represents (A*), (B*) or (C*) 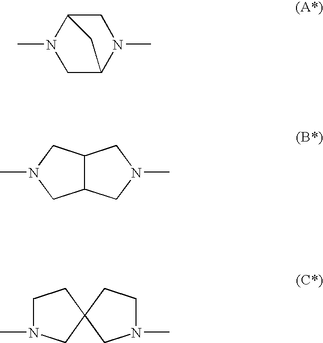
R¹ represents:
(a) -L-A wherein L is a bond or a C₁-C₂ alkyl moiety and A is a phenyl or indolyl group;
(b) -L-A-A′ wherein A′ is —O-A or —C(O)-A, L is a bond or a C₁-C₂ alkyl moiety and each A is the same or different and is a phenyl, pyridyl, tetrahydropyranyl or cyclopentyl group or -L-A-L-A wherein each A is a phenyl group and each L is the same or different and is a bond or a C₁-C₂ alkyl moiety;
(c) -A-Z-A wherein each A is the same or different and is a phenyl, pyridyl, thiazolyl, imidazolyl, tetrahydrofuranyl, tetrahydropyranyl, pyrrolidinyl, morpholinyl or pyrrolidinonyl group and Z is —O—(C₁-C₂ alkyl)-; and
(d) -A-Het-Y wherein A is a phenyl ring, Het is —O— and Y is -[L′-Het]ₙ-L′, -L′-B-L′ or -A-L-A, wherein n is 1 or 2, each L′ is the same or different and is a C₁-C₄ alkyl or C₂-C₄ alkenyl moiety, each Het is the same or different and is —O—, —NH— or —NMe-, B is —NH—CO—O—, each A is the same or different and is a phenyl or piperidinyl group and L is a C₁-C₂ alkyl moiety;
R² represents -L-A wherein L is a bond or a C₁-C₄ alkyl moiety and A is a phenyl, thienyl, pyridyl, dihydroindenyl or tetrahydronaphthalenyl group;
J is a direct bond, —NH— or —NMe-,
wherein:
the phenyl, heteroaryl, heterocyclyl and carbocyclyl groups and moieties in the groups R¹ and R² are unsubstituted or are substituted by one or two unsubstituted substituents which are the same or different and are selected from fluorine, chlorine, bromine, cyano, C₁-C₄ alkyl, C₂-C₄ alkenyl, C₁-C₂ haloalkyl, —O—(C₁-C₄ alkyl) or —O—(C₂-C₄ alkenyl); and
the alkyl, alkenyl and alkynyl groups and moieties in R¹ to R³ are unsubstituted or substituted by a single hydroxy or cyano substituent or by one, two or three fluorine substituents.

14. A compound according to claim 1, wherein said condition is chronic or acute pain, a bowel disorder, a bladder dysfunction, tinnitus or a demyelinating disease.

15. (canceled)

16. (canceled)

17. A pharmaceutical composition comprising a compound of the formula (I), as defined in claim 1, or a pharmaceutically acceptable salt thereof, and a pharmaceutically acceptable carrier or diluent.

18. A capsule or tablet comprising from 10 to 500 mg of a compound of the formula (I), as defined in claim 1, or a pharmaceutically acceptable salt thereof, and a pharmaceutically acceptable carrier or diluent.

19. An inhalation device comprising a pharmaceutical composition according to claim 17.

20. An inhalation device according to claim 19 wherein the device is a nebulizer.

21. A method of treating a patient suffering from or susceptible to a condition involving sodium ion flux through a sensory neurone specific channel of a sensory neurone, which method comprises administering to said patient an effective amount of a compound of formula (I), as defined in claim 1, or a pharmaceutically acceptable salt thereof.

22. The method of claim 21 wherein said condition is chronic or acute pain, a bowel disorder, a bladder dysfunction, tinnitus or a demyelinating disease.
